(19) United States
(12) Patent Application Publication
(10) Pub. No.: US 2010/0292302 A1
(43) Pub. Date: Nov. 18, 2010

(54) INDUCTION OF APOPTOSIS AND INHIBITION OF CELL PROLIFERATION THROUGH MODULATION OF CARNITINE PALMITOYLTRANSFERASE 1C ACTIVITY

(76) Inventors: Tak W. Mak, Toronto (CA); Guohua Pan, Oakville (CA); Yi Yao, Mississauga (CA); Kathrin Zaugg, Zo (CH)

Correspondence Address: LANDO & ANASTASI, LLP, ONE MAIN STREET, SUITE 1100, CAMBRIDGE, MA, 02142, US

(21) Appl. No.: 12/530,150

(22) PCT Filed: Mar. 7, 2008
(86) PCT No.: PCT/CA08/00448

§ 371 Date: Jun. 15, 2010

Related U.S. Application Data
(60) Provisional application No. 60/893,649, filed on Mar. 8, 2007.
(60) Provisional application No. 60/951,069, filed on Jul. 20, 2007.
(60) Provisional application No. 61/012,213, filed on Dec. 7, 2007.

(51) Int. Cl. A61K 31/7105; C07H 21/02; C12N 5/09; G01N 33/574; C12Q 1/68; A61P 35/00

(52) U.S. Cl. 514/44 A; 536/24.5; 435/325; 435/7.23; 435/6

(57) ABSTRACT

This invention relates to compositions and methods for cancer therapeutics. In particular, the present invention provides compositions and methods for treating tumors by inhibiting the activity of CPT1C. The methods and compositions can additionally include inhibition of glycolysis.

### RELATED APPLICATIONS

This application claims priority from the following patent applications:

U.S. Provisional Patent Application No. 60/893,649 filed Mar. 8, 2007;

U.S. Provisional Patent Application No. 60/951,069 filed Jul. 20, 2007; and

U.S. Provisional Patent Application No. 61/012,213 filed Dec. 7, 2007.

### FIELD OF THE INVENTION

This application relates to treatment of cancer through the reduction in the effective amount of CPT1C in tumor cells.

### BACKGROUND OF THE INVENTION

Normal cells that have sustained stress such as DNA damage activate the tumor suppressor p53 and thus are induced to either undergo apoptosis or arrest their cell cycles until the DNA is repaired {Vousden, K. H. p53: death star. Cell 103, 691-4 (2000)}. p53 is also a central factor in responses to a variety of other cellular stresses {J. G. Pan, T. W. Mak, Sci STKE 381, pe14 (2007)}. Metabolic stress leading to a real or threatened loss of energy capacity in a cell usually takes the form of ATP depletion due to glucose deprivation or hypoxia, respectively. This situation occurs frequently in rapidly growing solid tumors. Upon sensation of metabolic stress, most cancer cells execute a program whereby energy usage is limited and energy production is enhanced, particularly through glycolysis {J. S. Shaw, Current Opinion Cell Biol. 18, 598 (2006); 4. J. M. Brown, W. R. Wilson, Nat. Rev. Cancer 4, 437 (2004); Hue, L. et al. Insulin and ischemia stimulate glycolysis by acting on the same targets through different and opposing signaling pathways. J Mol Cell Cardiol 34, 1091-7 (2002); Wenger, R. H., Stiehl, D. P. & Camenisch, G. Integration of oxygen signaling at the consensus HRE. Sci STKE 2005, re 12 (2005)}. However, the role of p53 in this stress response is still controversial.

Although it has been suggested that hypoxic cells upregulate p53, two recent studies in which cDNA microarrays were used to compare gene expression patterns in p53-competent and p53-deficient cells revealed that only a handful of genes were regulated by p53 during hypoxia and very few of these were induced by p53 {Hammond, E. M. et al. Genome-wide analysis of p53 under hypoxic conditions. Mol Cell Biol 26, 3492-504 (2006); Liu, T. et al. Hypoxia induces p53-dependent transactivation and Fas/CD95-dependent apoptosis. Cell Death Differ 14, 411-21 (2007)}. Importantly, however, both papers demonstrated that p53 is indeed functional during hypoxia. Stronger evidence exists showing that p53 is activated during glucose deprivation and makes an important contribution to the ability of the cell to withstand metabolic stress {Jones, R. G. et al. AMP-activated protein kinase induces a p53-dependent metabolic checkpoint. Mol Cell 18, 283-93 (2005)}.

Fatty acid (FA) synthesis is an energy-depleting process required for cell proliferation, whereas fatty acid oxidation (FAO) is an oxygen-dependent catabolic process that supplies energy. Under conditions of glucose deprivation, FA synthesis is turned off in favour of FAO via inactivation of acetyl-CoA carboxylase (ACC) {Hardie, D. G., Hawley, S. A. & Scott, J. W. AMP-activated protein kinase—development of the energy sensor concept. J Physiol 574, 7-15 (2006)}. FAO has also been implicated in some cancers although little is known about its role in metabolic adaptation of tumor cells {Liu, Y. Fatty acid oxidation is a dominant bioenergetic pathway in prostate cancer. 9(3) 230-4 (2006); J. V. Swinnen, K. Brusselmans, G. Verhoeven, Current Opinion in Clinical Nutrition and Metabolic Care 9, 358 (2006)}. FAO is normally controlled at the step of FA import into the mitochondria {Ramsay, R. R. & Zammit, V. A. Carnitine acyltransferases and their influence on CoA pools in health and disease. Mol Aspects Med 25, 475-93 (2004); Liu, Y. Fatty acid oxidation is a dominant bioenergetic pathway in prostate cancer. 9(3) 230-4 (2006)}, a process regulated by the carnitine palmitoyltransferase 1 (CPT1) enzymes. There are three tissue-specific isoforms of CPT1: CPT1A, which is found in liver and other tissues, CPT1B predominantly in muscle, and CPTC1 in brain and testes. {J. Kerner, C. Hoppel, Biochimica et Biophysica Acta 1486, 1 (2000); N. T. Price et al., Genomics 80, 433 (2002)} Whereas CPT1A and CPT1B are enzymatically active, the CPT1C protein has not been shown to possess catalytic activity despite containing a carnitine acyltransferase structural motif and displaying high affinity malonyl-CoA binding {Price, N. et al. A novel brain-expressed protein related to carnitine palmitoyltransferase I. Genomics 80, 433-42 (2002); Wolfgang, M. J. et al. The brain-specific carnitine palmitoyltransferase-1c regulates energy homeostasis. Proc Natl Acad Sci USA 103, 7282-7 (2006)}. The precise physiological function of CPT1C has yet to be determined, although it has been proposed to act as an energy-sensing molecule involved in modulating malonyl-CoA levels in the CNS. Targeted gene disruption studies demonstrated that Cpt1c is not an essential gene in mice and Cpt1c-deficient animals have lower body weight and food intake, and exhibit reduced fatty acid oxidation {Wolfgang, M. J. et al. The brain-specific carnitine palmitoyltransferase-1c regulates energy homeostasis. Proc Natl Acad Sci USA 103, 7282-7 (2006)}. Paradoxically, Cpt1c-deficient mice show increased susceptibility to obesity when fed high-fat diet, suggesting that Cpt1c is protective against fat-induced obesity. It was thought that Cpt1c acts as an energy-sensing molecule involved in modulating malonyl-CoA levels in the CNS {Wolfgang, M. J. et al. The brain-specific carnitine palmitoyltransferase-1c regulates energy homeostasis. Proc Natl Acad Sci USA 103, 7282-7 (2006)}. Consistent with these findings, ectopic expression of Cpt1c in the CNS feeding centres protects mice from fat diet-induced body weight gain {Y. Dai, M. J. Wolfgang, S. H. Cha, M. D. Lane, Biochem. Biophy. Res. Comm. 359, 469 (2007)}. Targeted gene disruption studies demonstrated that Cpt1c is not an essential gene in mice and Cpt1c-deficient animals have lower body weight and food intake, and exhibit reduced fatty acid oxidation {Wolfgang, M. J. et al. The brain-specific carnitine palmitoyltransferase-1c regulates energy homeostasis. Proc Natl Acad Sci USA 103, 7282-7 (2006)}.

There is also a recent report in the literature of CPT1C being found to have carnitine palmitoyl transferase activity and to be localised in neurons but not in astrocytes of adult brain, and that CPT1C is localised in the ER of the cells and not in mitochondria. {Adriana Y Sierra, Esther Gratacós, Patricia Carrascol, Josep Clotet, Jesús Ureña, Dolors Serra, Guillermina Asins, Fausto G. Hegardt, Núria Casals. J. Biol. Chem., 10.1074/jbc.M707965200, published online Jan. 11, 2008} These researchers also demonstrated palmitoyl-CoA is a substrate of cpt1c.

### SUMMARY OF THE INVENTION

CPT1C was identified in a cDNA microarray screen as a potential novel p53 target gene that is upregulated in a p53-dependent manner in vitro and in vivo. Here, it was demonstrated that CPT1C expression increases fatty acid oxidation and protects cells from death induced by hypoxia or glucose deprivation. Furthermore, cells deficient in CPT1C have reduced ATP production, display mitochondrial defects and spontaneously succumb to apoptosis. CPT1C-depleted tumors were found to be significantly growth-suppressed in comparison to control tumors.

One embodiment of the invention is an isolated siRNA compound, at least a portion of which hybridizes to a CPT1C transcript under physiological conditions and decreases the expression of CPT1C in a cell.

In humans, the CPT1C transcript has a nucleotide sequence set forth in SEQ ID NO: 1. Preferably, the siRNA compound hybridizes to a coding sequence in SEQ ID NO: 1.

Typically, an siRNA compound of the invention is from about 14 to about 35 nucleotides in length, more preferably from about 18 to about 30 nucleotides in length.

The siRNA compound can be single-stranded, or it can be double-stranded.

An siRNA compound of the invention can include one or more modified backbone or base moieties.

The siRNA compound can be a hairpin RNA, and if so, the duplex portion is preferably from about 19 to about 24 nucleotides in length.

An siRNA compound of the invention can include an RNA strand containing SEQ ID NO: 8, 9, 10, or 11.

An siRNA compound of the invention can have one or more internucleotide linkage selected from alkylphosphonates, phosphorothioates, phosphorodithioates, alkylphosphonothioates, phosphoramidates, phosphate esters, carbamates, acetamidate, carboxylmethyl esters, carbonates, and phosphate triesters.

The invention includes a pharmaceutical composition comprising an siRNA compound of the invention in combination with a pharmaceutically acceptable carrier.

The invention includes a double stranded siRNA molecule that decreases expression of CPT1C gene, wherein each strand of said siRNA molecule is about 18 to about 30 nucleotides in length, and wherein one strand of said siRNA molecule contains a nucleotide sequence having sufficient complementarity to an RNA of said CPT1C gene for the CPT1C molecule to direct cleavage of said RNA via RNA interference (RNAi). Preferably, each strand includes at least about 14 to 24 nucleotides that are complementary to the nucleotides of the other strand. The siRNA molecule can be assembled from two separate oligonucleotide fragments wherein a first fragment includes the sense strand and a second fragment includes the antisense strand of the siRNA molecule. The sense and antisense strands can be coupled via a linker molecule. The linker can be a polynucleotide linker or a non-nucleotide linker.

The invention includes a method of decreasing CPT1C expression in a cell. This entails contacting the cell with an effective amount of an siRNA compound wherein the siRNA compound includes at least a portion that hybridizes to a CPT1C transcript under physiological conditions and decreases the expression of CPT1C in the cell.

In a related embodiment, the invention is a method of reducing the growth rate of a tumor cell. The method includes contacting the tumor cell with an siRNA compound in an amount sufficient to reduce the growth rate of the tumor. The siRNA compound includes at least a portion that hybridizes to a CPT1C transcript under physiological conditions and decreases the expression of CPT1C in the tumor cell.

Typically, the tumor cell expresses a higher level of CPT1C relative to a normal cell.

The invention includes a method for treating a tumor in a patient. The method includes administering to the patient an effective amount of an siRNA compound, wherein the siRNA compound has at least a portion that hybridizes to an CPT1C transcript under physiological conditions and decreases the expression of CPT1C in a tumor cell. Usually, the tumor cell expresses a higher level of CPT1C relative to a normal cell from a comparable tissue.

Often, the tumor is growing under hypoxic conditions. The determination of whether a tumor is growing under hypoxic conditions can be determined e.g. by measuring oxygen tension.

Tumors that can be treated include a lung tumor, brain tumor, prostate tumor, breast tumor, or colon tumor, preferably a solid tumor.

The invention includes also administering at least one additional anti-tumor chemotherapeutic agent that inhibits tumor cells. A preferred anti-tumor chemotherapeutic agent is a glycolysis inhibitor.

Another method for inhibiting tumor growth in a patient according to the invention includes administering to the patient an effective amount of an siRNA compound, wherein the siRNA compound comprises at least a portion that hybridizes to an CPT1C transcript under physiological conditions and decreases the expression of CPT1C in a tumor cell.

The invention includes use of an siRNA molecule of the invention in the manufacture of medicament for the treatment of a tumor.

Methods of the invention include use of an antisense molecule.

Typical agents of the invention down-regulate CPT1C so as to at least lower, and preferably eliminate, the protective effect against cell death in tumor cells that the CPT1C protein exerts.

The invention includes a method for treating tumor cells in an individual suffering from a cancer that expresses CPT1C in amounts higher than in normal tissue of the same type, comprising administering to the individual a composition effective to inhibit expression of CPT1C by the tumor cells and increase apoptosis in the tumor cells.

Another embodiment is a method for treating tumor cells in an individual suffering from a cancer that depends on CPT1C for survival under hypoxic conditions. The method includes administering to the individual a composition effective to inhibit expression of CPT1C by the tumor cells and increase apoptosis or reduce proliferation in the tumor cells.

A particular embodiment of the invention is a method for treating a cancer patient, that includes (a) identifying cancer cells in the patient that have upregulated expression of CPT1C; and (b) administering to the individual a composition effective to inhibit expression of CPT1C by the cancer cells.

Preferably, the cancer is lung cancer and the composition which inhibits expression of CPT1C comprises an antisense oligonucleotide complementary to a portion of an mRNA encoding CPT1C. The oligonucleotide can be from 8 to 80 bases in length, more preferably from 9 to 70, more preferably between 10 and 60, or 11 and 50, or 12 and 40, or from is 15 to 30 bases in length.

Another method is for treating a cancer patient that includes: (a) identifying cancer cells in the patient that contain a level of a substance associated with higher than normal CPT1C activity; and (b) administering to the individual a composition effective to inhibit expression of CPT1C by the cancer cells.

A slightly different approach according to the invention is a method for treating cancer in a subject that includes administering a nucleic acid having a promoter operatively linked to a nucleic acid sequence of interest wherein the promoter is known to be up-regulated in cancer cells, and wherein the nucleic acid sequence of interest down-regulates expression of CPT1C resulting in growth suppression or death of the cancerous cells.

The nucleic acid has can have a sequence complementary to at least a portion of the sequence consisting of GGGCAGGCGAGTAGGGCTTCTCCATCACTTGTCCTGGACATGCCT (SEQ ID NO:6). Typically, the sequence is at least 25 nucleotides in length. The administration step can be carried out via injection, oral administration, topical administration, adenovirus infection, liposome-mediated transfer, topical application to the cells of the subject, or microinjection.

Such method can also includes inhibiting glycolysis in cancer cells. Typically this is achieved by administering a glycolysis inhibiting agent. Preferably, the glycolysis inhibiting agent is 2-deoxyglucose, lonidamine, 3-bromopyruvate, imatinib or oxythiamine, or a mixture of two or more or these. A preferred glycolysis inhibiting agent is 3-bromopyruvate.

The invention also includes a method of screening for compounds that down-regulate expression of CPT1C. Such method can include: a) contacting a nucleic acid molecule that has a promoter from a CPT1C gene operatively linked to a reporter gene with a candidate compound; and b) assessing the level of expression of the reporter gene. The method can be carried out in a cell free system, a cell or a tissue.

The nucleic acid molecule may be in the form of a non-viral vector.

The step of assessing the level of expression of the reporter gene typically includes measuring the level of mRNA transcribed from the reporter gene, or it can include measuring the level of protein translated after transcription of the reporter gene.

The method can include the further steps of administering to a mammal suffering from cancer a candidate compound found to down-regulate expression of CPT1C, and assessing its effect growth of the cancer.

According to yet another embodiment, the invention is a method for identifying a potential anti-cancer agent which comprises: (a) contacting a cell with the agent wherein the cell comprises a nucleic acid comprising a CPT1C promoter operatively linked to a reporter gene; (b) measuring the level of reporter gene expression in the cell; and (c) comparing the expression level measured in step (b) with the reporter gene expression level measured in an identical cell in the absence of the agent, wherein a lower expression level measured in the presence of the agent is indicative of a potential anti-cancer agent.

Preferably, the cell is a cancer cell, often a melanoma cell, a neuroblastoma cell, a cervical cancer cell, a breast cancer cell, a lung cancer cell, a prostate cancer cell, a colon cancer cell or a glioblastoma multiforme cell.

Again, the agent can be an antisense nucleic acid including a nucleotide sequence complementary to at least a portion of the sequence consisting of GGGCAGGCGAGTAGGGCTTCTCCATCACTTGTCCTGGACATGCCT (SEQ ID NO:6). The agent can be a DNA molecule, a carbohydrate, aglycoprotein, a transcription factor protein or a double-stranded RNA molecule. The reporter gene might encode, for instance, β-galactosidase, luciferase, chloramphenicol transferase or alkaline phosphatase.

The invention includes a method for identifying a potential anticancer agent comprising: (i) operatively linking a CPT1C promoter with a reporter gene of interest; (ii) introducing the resulting expression cassette into a target cell; (iii) contacting the target cell with a candidate agent; and (iv) comparing the level of reporter gene expression in the presence and absence of the agent, wherein a potential anticancer agent is one that produces a measurable decrease in the level of reporter gene expression in the presence of the agent. The reporter gene can be a β-galactosidase gene, a β-glucuronidase gene, a β-lactamase gene, an alkaline phosphatase gene, a gene encoding secreted alkaline phosphatase, a chloramphenicol aminotransferase gene, a luciferase gene, or a gene encoding a fluorescent protein.

In a related embodiment for identifying a potential anticancer agent, a method includes: (i) operatively linking a p53-responsive element of an intronic sequence of a CPT1C gene with a reporter gene of interest; (ii) introducing the resulting expression cassette into a target cell; (iii) contacting the target cell with a candidate agent; and (iv) comparing the level of reporter gene expression in the presence and absence of the agent, wherein a potential anticancer agent is one that produces a measurable decrease in the level of reporter gene expression in the presence of the agent. Again, the reporter gene can be a β-galactosidase gene, a β-glucuronidasegene, a β-lactamase gene, an alkaline phosphatase gene, a gene encoding secreted alkaline phosphatase, a chloramphenicol aminotransferase gene, a luciferase gene, or a gene encoding a fluorescent protein.

Yet another method for identifying a potential anticancer agent includes: (i) operatively linking a p53-responsive element of an intronic sequence of a CPT1C gene with a reporter gene of interest; (ii) introducing the resulting expression cassette into a target cell; (iii) contacting the target cell with a candidate agent under conditions in which p53 is produced or is present in the cell; and (iv) comparing the level of reporter gene expression in the presence and absence of the agent, wherein a potential anticancer agent is one that produces a measurable decrease in the level of reporter gene expression in the presence of the agent.

Another method for screening and identifying a compound that is capable of decreasing cellular levels of CPT1C includes: a) exposing cells to the compound to be screened; and b) determining whether the compound acts upon the DNA motifs that regulate the CPT1C gene, wherein the +1 position is the transcription start of the gene, to decrease gene expression, thereby identifying a compound capable of decreasing cellular levels CPT1C.

Another aspect of the invention is a method of screening for a candidate substance as an anticancer agent that regulates activity of the CPT1C promoter in which the method includes a step selected from the group consisting of: (a) contacting a nucleic acid comprising a CPT1C promoter with a CPT1C promoter binding protein and the candidate substance under conditions that allow binding between the protein and the promoter and determining whether the candidate compound modulates the binding between the protein and the promoter; and (b) contacting the candidate substance with a cell comprising the CPT1C promoter operably attached to a reporter gene coding for an expression product and assaying for expression of the reporter gene expression product. Preferably, the protein is p53.

Another method of screening anti-cancer agents for treating a human comprises: (a) contacting a mammalian CPT1C protein with a test agent thought to be effective in inhibiting the activity of said protein in the presence of a fatty acyl-CoA known to be a substrate of said protein; (b) determining if said test agent inhibits the activity of said protein, wherein determining if said test agent inhibits the activity of said protein comprises quantitating the amount of fatty acyl-carnitine produced in the presence of said agent; and (c) classifying said test agent as a potential anti-cancer agent if said test agent inhibits the activity of said protein.

In a particular embodiment, the known substrate is palmitoyl-CoA, the source of which can be palmitic acid.

Such method can further include determining if the test agent inhibits the activity of CPT1A in the presence of the substrate, wherein if the inhibition of CPT1C is greater than the inhibition of CPT1A by at least 3 fold, and then (d) classifying said test agent as an anti-cancer agent.

There can be another step of (e) determining whether an agent classified as an anti-cancer agent in step (d) inhibits in vitro growth of human tumor cells.

If the agent is determined to inhibit in vitro growth of human tumor cells, then the method can also include the step of (f) determining whether the agent inhibits tumor growth in a non-human mammal. The invention includes use of an agent determined to inhibit tumor growth in step (f) in clinical trials for the treatment of cancer, and further in the treatment of cancer in humans.

### BRIEF DESCRIPTION OF THE DRAWINGS

FIG. 1. CPT1C is a p53 target gene that is upregulated by p53 in vivo. A, B. Temperature-sensitive activation of p53 in DP16.1/p53ts cells. DP16.1 (control; p53−/−) and DP16.1/p53ts (p53ts) cells were cultured for 6 hrs at 37° C. or 32° C. A temperature shift from 37° C. to 32° C. activates p53 in DP16.1/p53ts cells. A. Total RNA (10 μg) from these cells was subjected to Northern blotting. Full-length Cpt1c cDNA was used as the probe. P21 and Pidd were used as positive controls for p53 activation. B. Cells from the cultures in A were subjected to real time RT-PCR using Sybr Green to detect the indicated CPT family members. P21, positive control. Values shown are fold induction normalized to Gapdh expression. C. Induction of CPT1C expression by DNA damaging and chemotherapy agents. MCF-7 cells were treated with the indicated DNA-damaging stimuli as described in Materials and Methods and real-time RT-PCR was performed to detect expression of the indicated CPT family members. Damage-inducible gene p21 was used as positive control. All values shown were normalized to GAPDH and fold induction was calculated relative to the untreated control. Similar results were obtained for cell lines U87 and A549 (data not shown). D. p53 upregulates Cpt1c in vivo. E12.5 C57/b16 embryos from p53+/− and p53−/− mice were subjected to 5 Gy ionizing radiation in utero. Embryos were harvested and prepared for in situ hybridization at 8 hrs post-irradiation. Incubation of midbrain sections with a Cpt1c riboprobe showed that Cpt1c mRNA was upregulated in irradiated p53+/− embryos (bottom panel) compared to sham-irradiated controls (top panel), but not in irradiated or sham-irradiated p53−/− cells. For all Figures, results shown are one trial representative of at least 3 independent experiments. E. the nucleotide sequence CPT1C mRNA (SEQ ID NO: 1) (GenBank sequence nm_{—}152359).

FIG. 2. Localization of CPT1C in the mitochondria. The full-length mouse CPT1C cDNA was FLAG-tagged at the C-terminus and transiently expressed in HeLa cells. Confocal microscopy showed that FITC-labelled anti-flag antibody co-localized with Mitotracker Red CMXRos (Eugene, Oreg., USA) in the mitochondria.

FIG. 3. p53 directly activates CPT1C transcription. A. p53 binding sites. Computational analysis revealed two putative p53-responsive elements, p53-RE-A and p53-RE-B, located in intron 1 in the murine Cpt1c promoter region as indicated. B. p53 binding to p53-RE-A. ChIP analysis was performed on DP16.1/p53ts cells cultured at 37° C. or 32° C. Only in cells maintained at 32° C. did p53 bind directly to the first intron of Cpt1c. The proximity of p53RE-A and -B makes it difficult to determine precisely where p53 is binding. Un-precipitated genomic DNA was used as loading control. N.A, no p53 antibody. C. p53-RE-A binds to p53 and activates transcription. The indicated luciferase reporter contructs were transfected into E1A/Ras-transformed p53^{-/-} MEFS, with or without cotransfection of WT p53 or a DNA-binding mutant of p53 (p53*). Relative luciferase activity was taken as the relative transcriptional activity. pGL3-SV40, vehicle control; p53-RE-A*, mutated p53-RE-A (G→*T at position 42) unable to support p53-dependent transcription. p53-RE-B could not stimulate transcription in a p53-dependent manner under any circumstances.

FIG. 4. CPT1C expression is induced by hypoxia in cells and tumors and up-regulated in human lung cancers. A. Upregulation in hypoxic ES cells. WT ES cells were treated with 0.2% hypoxia for 24 hrs and levels of Cpt1a, Cpt1b, Cpt1c and Cpt2 mRNAs were measured in total RNA using real time PCR. mRNA levels were normalized to Gapdh expression. Results shown are the fold induction expressed as a ratio of the values obtained under hypoxia over those obtained under normoxia. Vegf, positive control. RI, RNAse inhibitor as a reference control. B. Dependence on p53. p53^{+/+} (wt) and p53^{-/-} (null) MEFs were treated with 0.2% hypoxia for 7 hrs and levels of Cpt1a, Cpt1b, Cpt1c and Cpt2 mRNAs were measured and results expressed as for A.

FIG. 5. Induction of CPT1C expression by hypoxia. Breast (A, C, E), lung (B, C) and colon (D) cancer cell lines were cultured at 24 hrs post-seeding in 0.2% O₂ for 24, 48 or 72 hrs and CPT1C mRNA was measured using real-time RT-PCR. Relative expression values shown are CPT1C mRNA expression levels normalized to β-actin mRNA. F. HCT116 p53^{+/+} and HCT116 p53^{-/-} colon cancer cells were treated 24 hrs after seeding with 0.2% hypoxia for 48 hrs and CPT1C mRNA was measured using real time RT-PCR. Values shown are CPT1C mRNA expression levels normalized to β-actin mRNA. G. Breast and colon cancer cell lines exposed to hypoxia (0.2%) for 24 hrs. The total RNAs were then prepared. The CPT1C transcript levels were determined by real time RT-PCR. (“+” indicates hypoxia, “−” indicates controls (normoxia). The RT-PCR primers used are (forward primer): GCC ATG GAG GAC AAA GAG AA (SEQ ID NO: 2) and (reverse primer) ACG ATG TAC AGC GCA AAC AG (SEQ ID NO:3). CPT1C mRNA expression was found to be significantly induced by hypoxia. H. Confirmation of murine tumor hypoxia. Tumor-bearing PyMT mice were injected with the extrinsic hypoxia marker EF5 and subjected to chronic hypoxia (+; see Materials and Methods) or normoxia (−). Tumors isolated from these animals were immunostained to detect EF5, a hypoxia marker. Results are the mean percentage±S.E. of the tumor area that was hypoxic (stained positively for EF5) for 4 tumor samples from controls and 5 samples from hypoxic animals.

FIG. 6. Induction of CPT1C expression by hypoxia. Upregulation of Cpt1c in hypoxic tumors. Tumors from the animals in FIG. 5(F) were examined by bright field and dark field microscopy. (a, b) Tumors from normoxic controls show background Cpt1c expression. (c, d) Tumors from animals exposed to chronic hypoxia show elevated Cpt1c expression. (e, f) Cpt1c expression was mainly restricted to tumor cells (arrows). Scale bar, 100 (micrometers).

FIG. 7. CPT1C expression in lung tumors. Elevation of CPT1C in human lung tumors. Levels of CPT1A, CPT1B, CPT1C and HIF1A mRNAs were determined by real-time RT-PCR in human lung tumor samples and matched normal lung tissue samples. Results shown are the fold change in a given mRNA in tumor tissue compared to matched normal tissue from the same patient.

FIG. 8. CPT1C expression in various cancer cell lines and lung tumors.

Levels of CPT1C mRNA were measured in cell lines derived from breast cancer (A), lung cancer (B), prostate cancer (C) and brain cancer (D) and their corresponding normal cell lines or primary normal cells using real time RT-PCR. For breast cancer cell lines, both normal cell lines (184A1 and 184B5) and human primary mammary epithelial cells (HMEC) were used as controls. For lung cancer cell lines, normal human bronchial epithelial (NHBE) and small airway epithelial cells (SAEC) were used as controls. For prostate cancer cell lines, normal prostate epithelial cells (PrEC) were used as controls. The expression levels were normalized over those of β actin. Relative expression was shown.

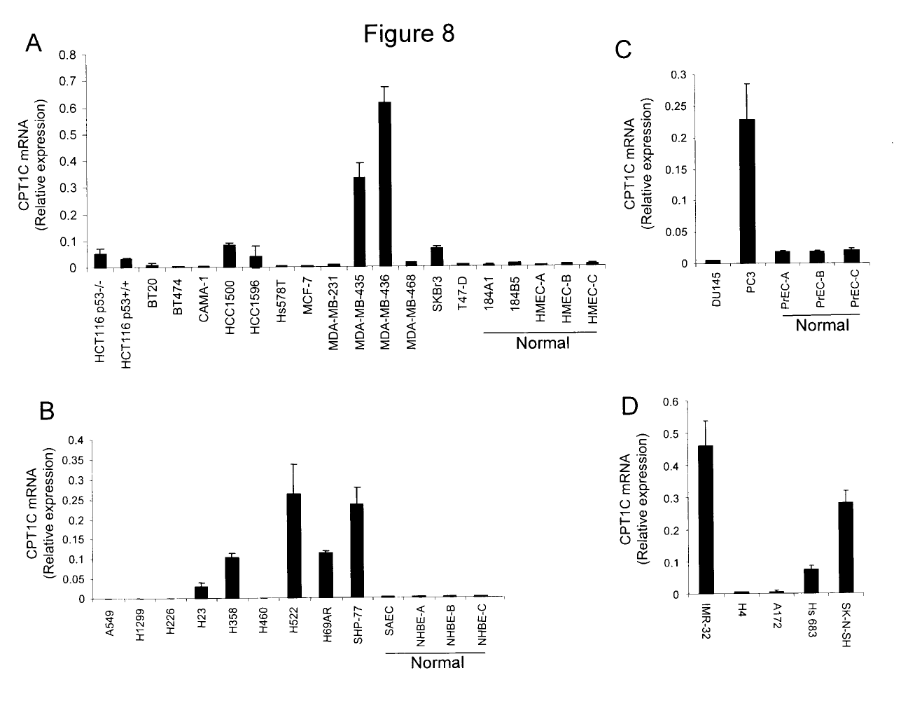

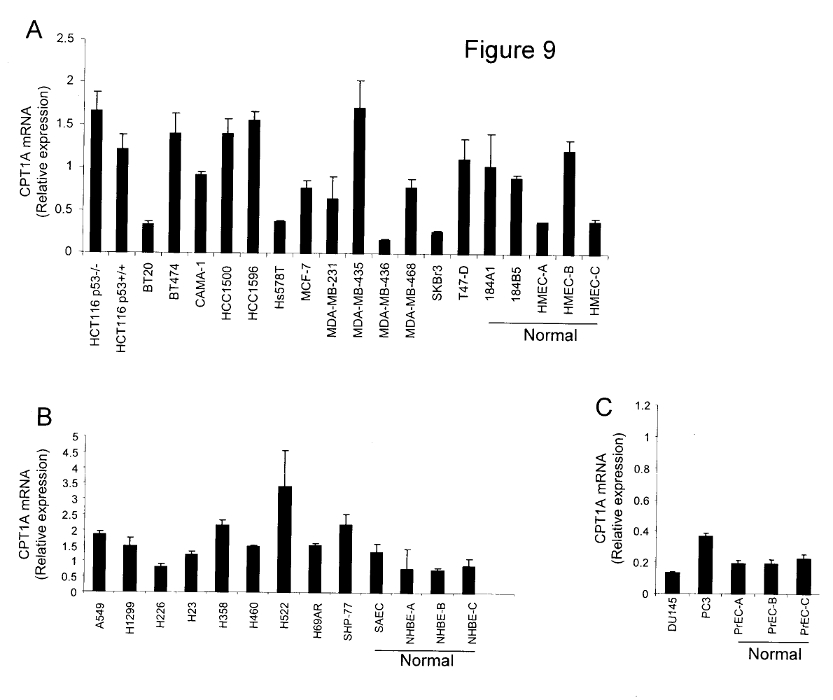

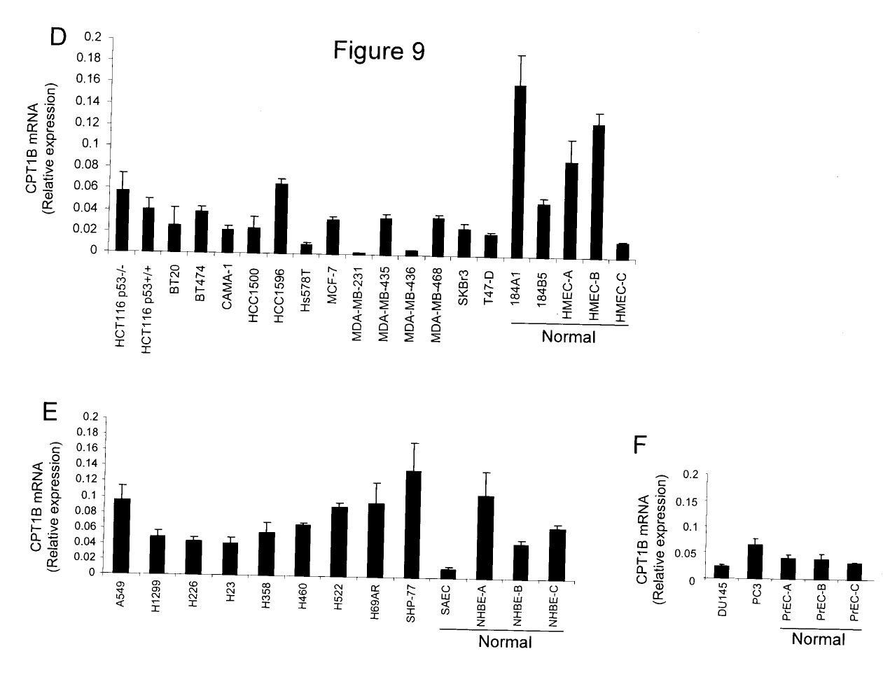

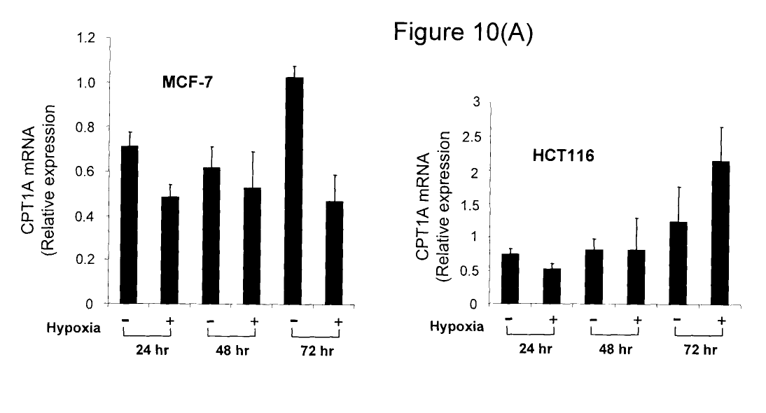

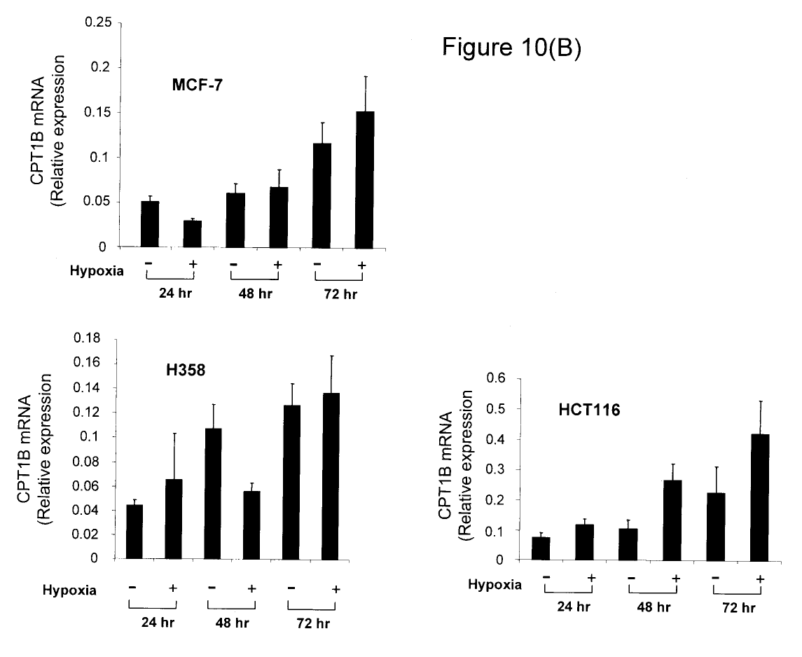

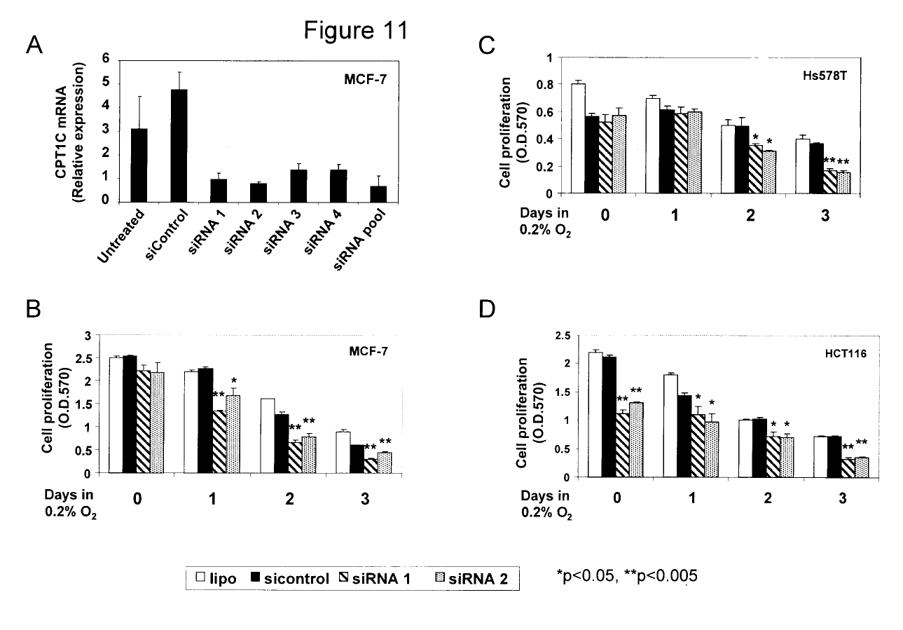

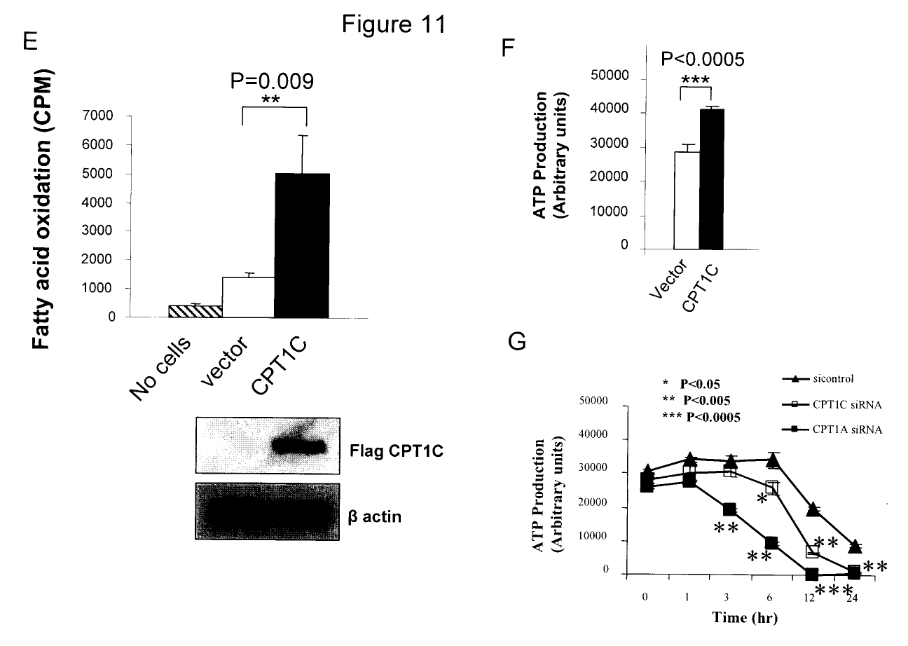

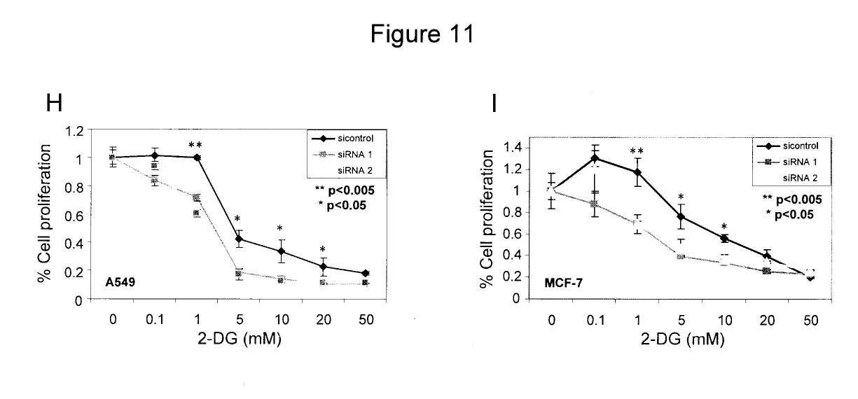

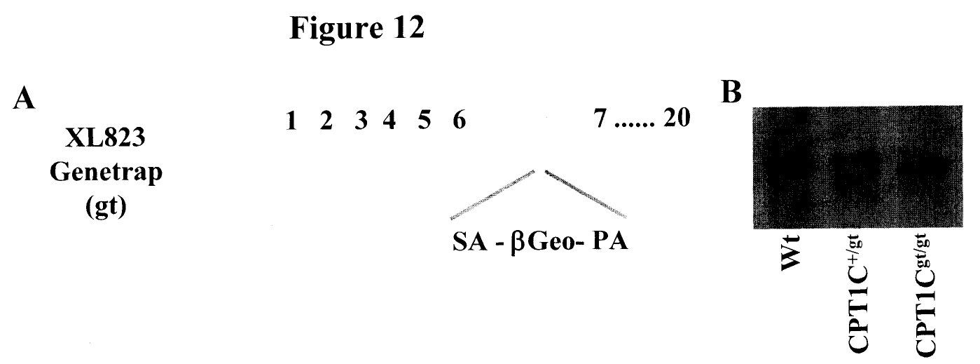

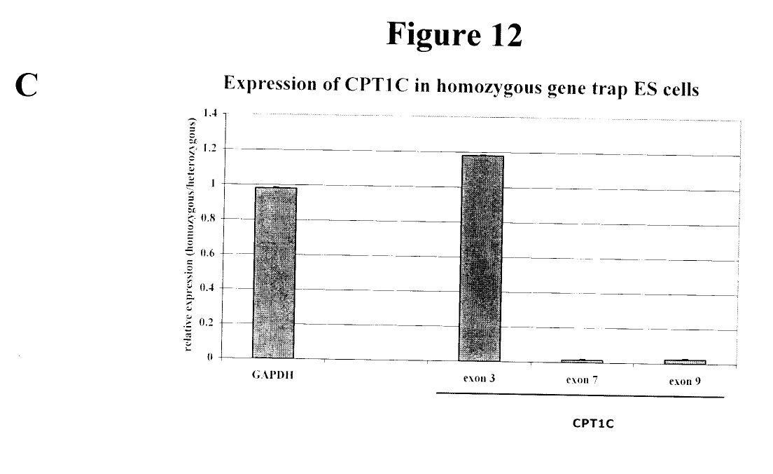

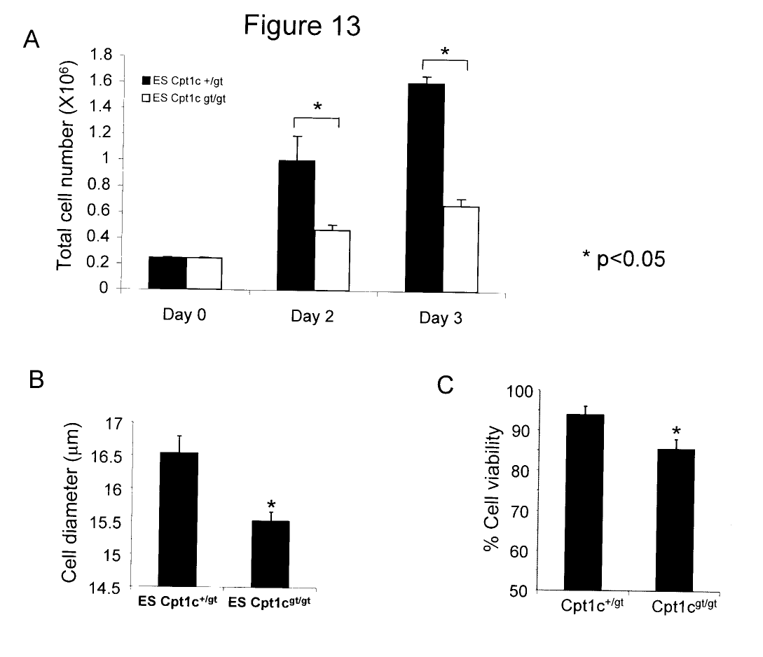

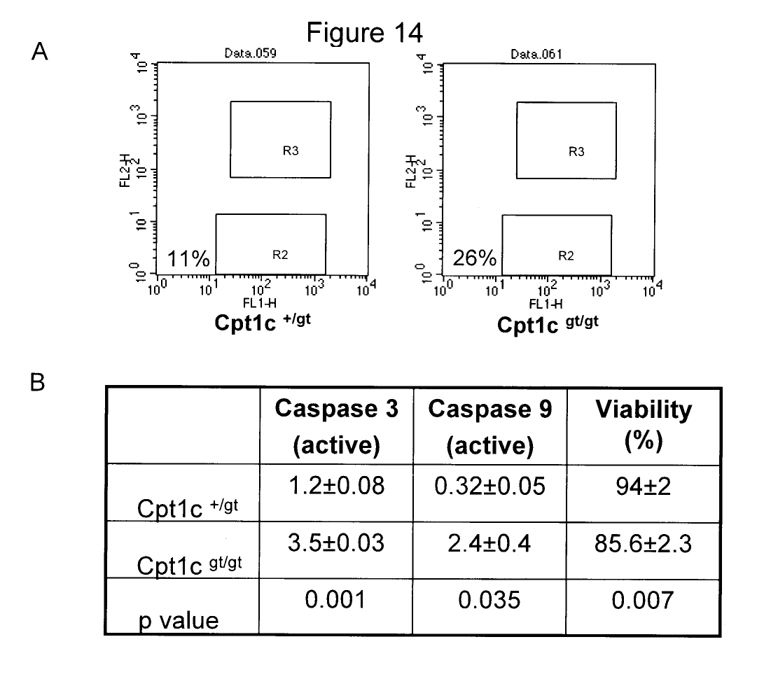

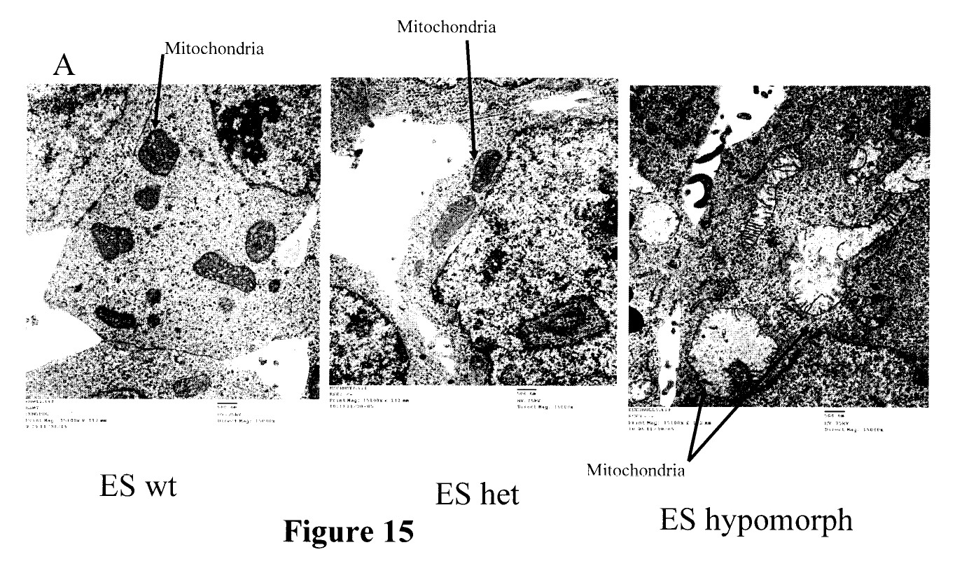

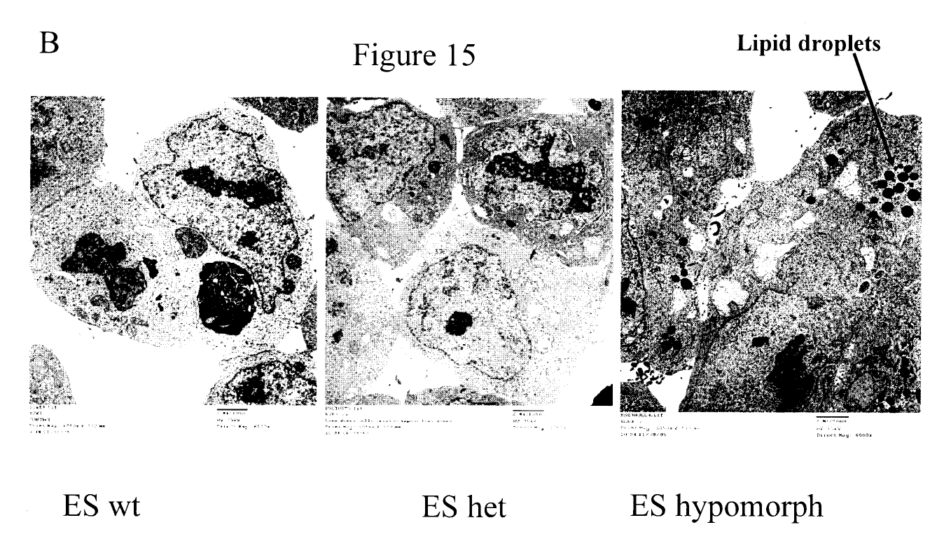

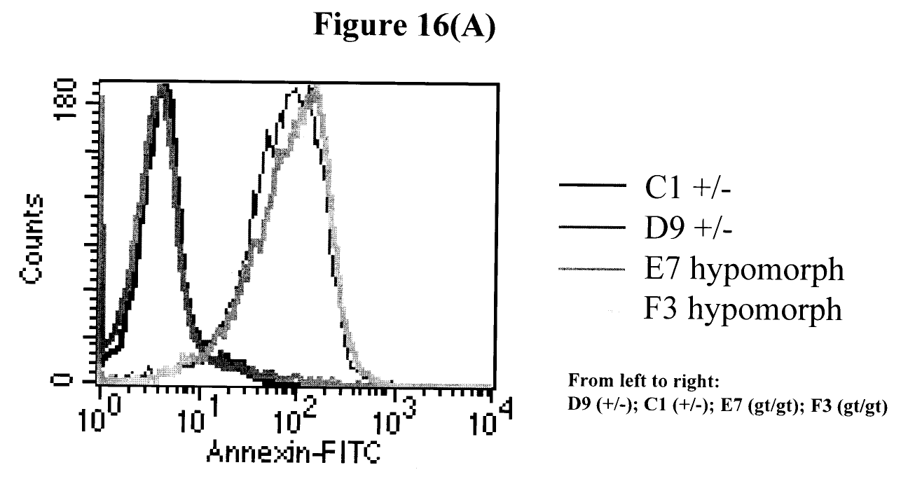

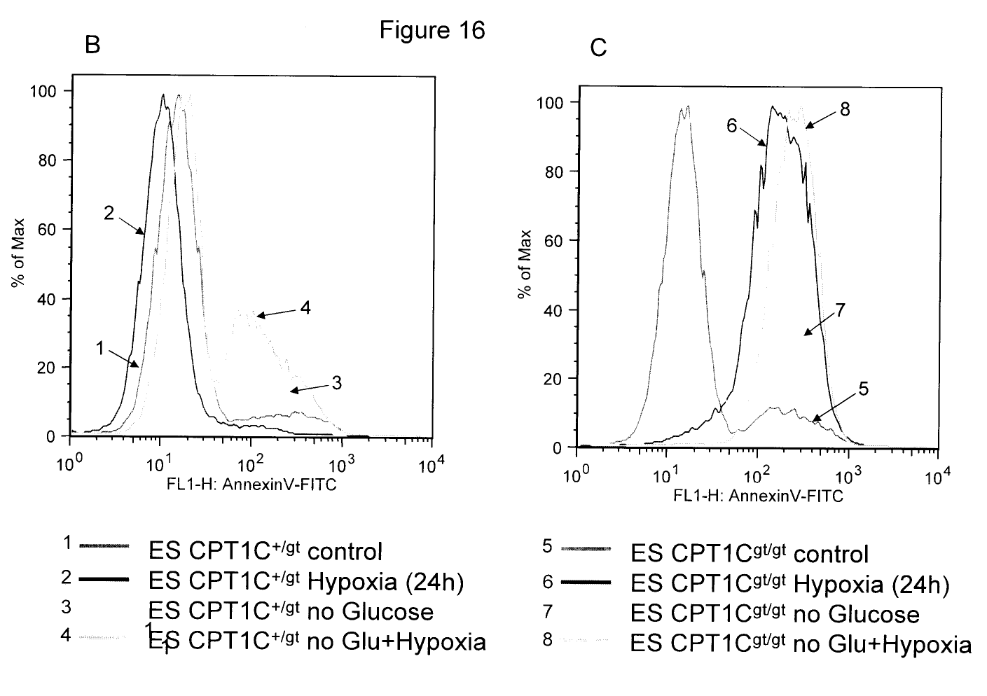

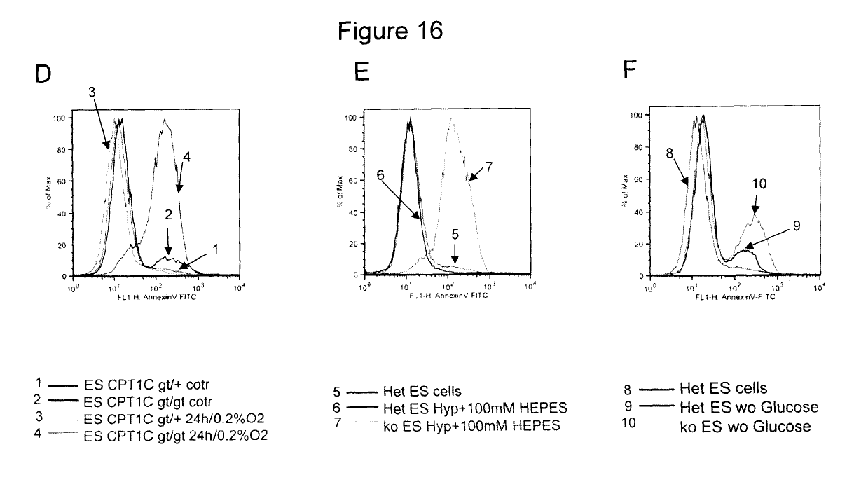

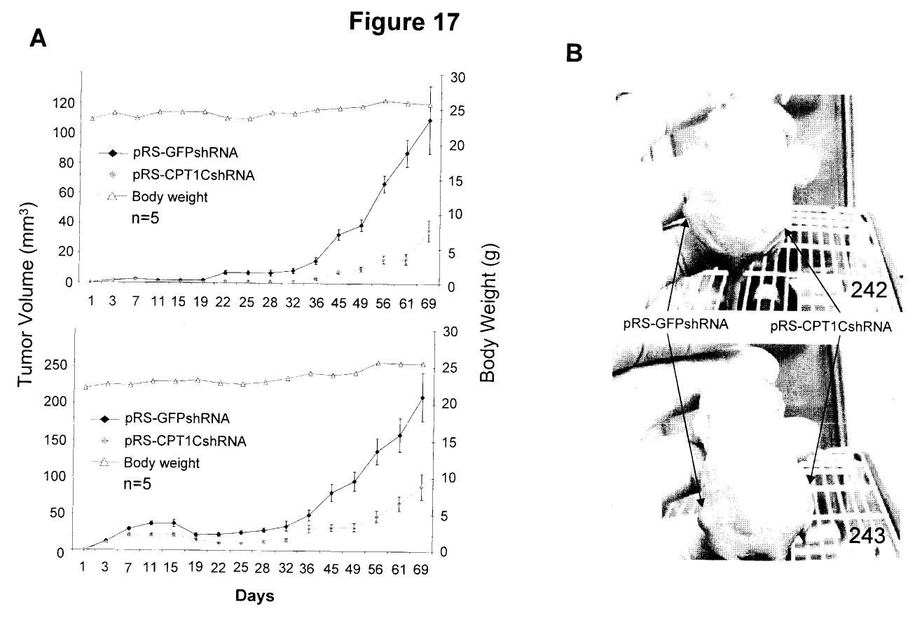

FIG. 9. Expression of CPT1A and CPT1B in cancer cell lines. Levels of CPT1A (A-C) and CPT1B (D-F) mRNA transcripts were measured quantitative RTPCR in various cancer cell lines. Their corresponding normal cell lines or primary cells were used as comparative controls as described in the legend to FIG. 8.

FIG. 10. Expression of CPT1A and 1B in cancer cell lines subjected to hypoxia. Breast (MCF7), lung (H358) and colon (HCT116) cancer cell lines were cultured at 24 hrs post-seeding in 0.2% O₂ for 24, 48 or 72 hrs and CPT1A and CPT1B mRNA levels were measured using real-time RT-PCR. Relative expression values shown are CPT1A and 1B mRNA expression levels normalized to β-actin mRNA.

FIG. 11. Effect of CPT1C depletion on cell growth, FAO and ATP production. A. CPT1C knockdown in MCF-7 cells. MCF-7 cells were left untreated, or treated with a non-silencing luciferase siRNA (sicontrol), or with one of 4 siRNAs targeting four sequences specific for CPT1C (siRNA 1-4), or with a pool of these 4 siRNAs. At 72 hrs post-transfection, CPT1C mRNA expression was measured using real-time RT-PCR and normalized to β-actin expression. B. C. D. siRNA knockdown of CPT1C reduces the proliferation of hypoxic cancer cell cultures. MCF-7 (B), Hs578T (C) and HCT116 (D) cells were transfected with Lipofectamine 2000 alone (lipo), with luciferase siRNA (sicontrol), or with one of the two CPT1C siRNAs (siRNA1 or 2). Transfected cells were cultured in 0.2% O₂ for 0, 1, 2 or 3 days prior to incubation under normoxia for 5, 4, 3 or 2 days, respectively. Cell growth was then measured using SRB staining. Results shown are the mean proliferation±SD of triplicate samples expressed as OD₅₇₀. E. F. G. Constitutive CPT1C expression increases FAO and ATP. MCF-7 cells were stably transfected with control vector or vector expressing FLAG-tagged CPT1C and evaluated for FAO (E) and ATP (F) (see Materials and Methods). The expression of FLAG-CPT1C was confirmed by Western blotting. Results shown are the mean cpm±SD of triplicate cultures. (G) Depletion of CPT1C reduces ATP production. PC3 cells were transfected with either CPT1C, or sicontrol siRNAs and cultured in glucose-free medium. ATP production was measured as described in Materials and Methods. Results are expressed as the mean ATP production±SD of triplicate samples. H. I. siRNA knockdown of CPT reduces the proliferation of cancer cells treated with 2-DG. A549 (H) and MCF-7 (I) cells were cultured for 24 hrs in 96-well plates in DMEM containing high glucose (20 mM) prior to transfection with sicontrol, CPT1C siRNA1 or CPT1C siRNA2 as indicated. The indicated concentrations (mM) of the glycolytic inhibitor 2-DG were added to wells at 24 hrs post-transfection and cells were cultured for 5 days. Cell growth was measured using SRB staining. Results shown are the mean growth±SD of triplicate samples expressed as OD₅₇₀ and compared to cultures with no 2-DG treatment. Student's t tests were performed by comparing the proliferation of sicontrol and CPT1C knockdown cells at the same 2-DG concentration.

FIG. 12. Generation and characterization of Cpt1c^{+/gt} and Cpt1c^{gt/gt} cells. A. Gene trap (gt) Cpt1c allele in ES cell clone XL823 (BayGenomics). A vector containing a splice acceptor site, β-Geo and a polyadenylation site (PA) is integrated into intron 6 of the murine genomic Cpt1c gene. B. Generation of Cpt1c^{+/gt} and Cpt1c^{gt/gt} cells. XL823 cells were cultured in 8 mg/ml G418 and surviving clones were analyzed by Southern blot analysis to determine heterozygosity (Cpt1c^{+/gt} cells) or homozygosity (Cpt1c^{gt/gt} cells) for the gt allele. C. Expression of CPT1C in CPT1C^{+/GT} and CPT1C^{GT/GT} cells. Real time PCR using primer sets corresponding to exons 3, 7 and 9 of the CPT1C gene were used to detect expression of CPT1C mRNA. Results shown are the level of the full-length transcript in CPT1C^{GT/GT} cells relative to its level in CPT1C^{+/GT} cells and are one trial representative of three trials.

FIG. 13. Cpt1c^{gt/gt} cells show decreased cell number, diameter and viability. A. Decreased cell number. Cpt1c^{+/gt} and Cpt1c^{gt/gt} cells were cultured for 3 days under standard conditions and total cell numbers were determined on the indicated days by counting using a Coulter Vi-Cell XR (Beckman) cell counter. Results are expressed as the mean total cell number (x10⁶)±S.D. of triplicate samples. B. Decreased cell diameter. The diameters of Cpt1c^{+/gt} and Cpt1c^{gt/gt} cells were measured using a Coulter Vi-Cell X R(Beckman) cell counter. Results shown are the mean cell diameter±S.D for triplicate samples of approx 10⁶ cells. C. Decreased viability. Cpt1c^{+/gt} and Cpt1c^{gt/gt} cells were cultured under standard conditions for one day, stained with Annexin V, and analyzed by flow cytometry to detect apoptotic cells. Values shown are the mean % viability±S.D. of at least 3 samples per genotype.

FIG. 14. Cpt1c^{gt/gt} cells show decreased mitochondrial membrane potential and spontaneous activation of the mitochondrial apoptosis pathway. A. Decreased mitochondrial membrane potential. Cpt1c^{+/gt} and Cpt1c^{gt/gt} ES cells were cultured under normal conditions and mitochondrial membrane potential was measured by flow cytometry using JC-1. Results shown are one trial representative of 3 experiments. B. Increased caspase-3 and -9 activation. The cells in A were analyzed for the presence of activated caspase-3 and -9 as described in Materials and Methods. Viability was determined by Annexin V staining. FIG. 15. Mitochondrial and cellular abnormalities of CPT1C^{GT/GT} ES cells. A. Three electron micrographs, wild type, heteromorph, and hypomorph, respectively, from left to right, illustrating morphology of the cells at 15,000× magnification. The CPT1C^{GT/GT} ES cells show a significant swelling of the mitochondria with a highly abnormal internal membrane structure. B. Morphology of the cells is shown. In comparison to CPT1C^{GT/GT} cells, CPT1C^{GT/GT} cells showed cytoplasmic lipid droplets and swollen mitochondria that had lost their internal structure. Electron micrographs taken at 6000× magnification. CPT1C^{GT/GT} ES cells show accumulation of cytoplasmic lipid droplets that were not detectable in heterozygous ES cells or wild-type ES cells.

FIG. 16. Generation and characterization of Cpt1c^{+/gt} and Cpt1c^{gt/gt} cells and sensitivity of CPT1C^{GT/GT} ES cells to hypoxia. A. CPT1C^{+/GT} and CPT1C^{GT/GT} cells were cultured for 24 hrs under hypoxic conditions (0.2% oxygen). Apoptosis was detected using Annexin V staining and flow cytometry. Results from one trial, representative of five experiments, are shown. B. C. CPT1C-deficient ES cells are more sensitive to hypoxia or glucose deprivation. CPT1C^{+/GT} (B) or CPT1C^{GT/GT} (C) ES cells were subjected to hypoxia (0.2%, 24 hrs), no glucose or both hypoxia and no glucose. The cell viability was then determined with Annexin V staining and flow cytometry analysis. D. Increased apoptosis of hypoxic Cpt1c^{gt/gt} cells. Cpt1c^{+/gt} and Cpt1c^{gt/gt} cells were cultured for 24 hrs under normoxia (control) or hypoxia (0.2% O₂) and apoptosis was detected using Annexin V staining and flow cytometry. E. Hypoxia-induced acidosis does not cause the death of Cpt1c^{gt/gt} cells. Cpt1c^{+/gt} and Cpt1c^{gt/gt} ES cells were treated as in (D) with the addition of 100 mM HEPES. Apoptosis was measured as for (D). F. Increased apoptosis of Cpt1c^{+/gt} cells subjected to glucose withdrawal. Cpt1c^{+/gt} and Cpt1c^{gt/gt} ES cells were cultured for 48 hrs in standard DMEM or DMEM lacking glucose (wo glucose) Apoptosis was measured as for (D).

FIG. 17. CPT1C depletion suppresses tumor growth in xenograft model. A. MDA-MB-468 breast cancer cells infected with retroviruses harboring shRNA targeting CPT1C (pRS-CPT1C shRNA) or a control gene (pRS-GFP shRNA) were injected subcutaneously into the left and right hindlimb, respectively, of nude mice at concentrations of 1.25×10⁶ or 5×10⁶ cells. B. The tumors were measured and tumor volumes were calculated twice a week for approximately 10 weeks. The sizes of representative tumors from two mice are shown as examples as indicated.

### DETAILED DESCRIPTION OF THE INVENTION

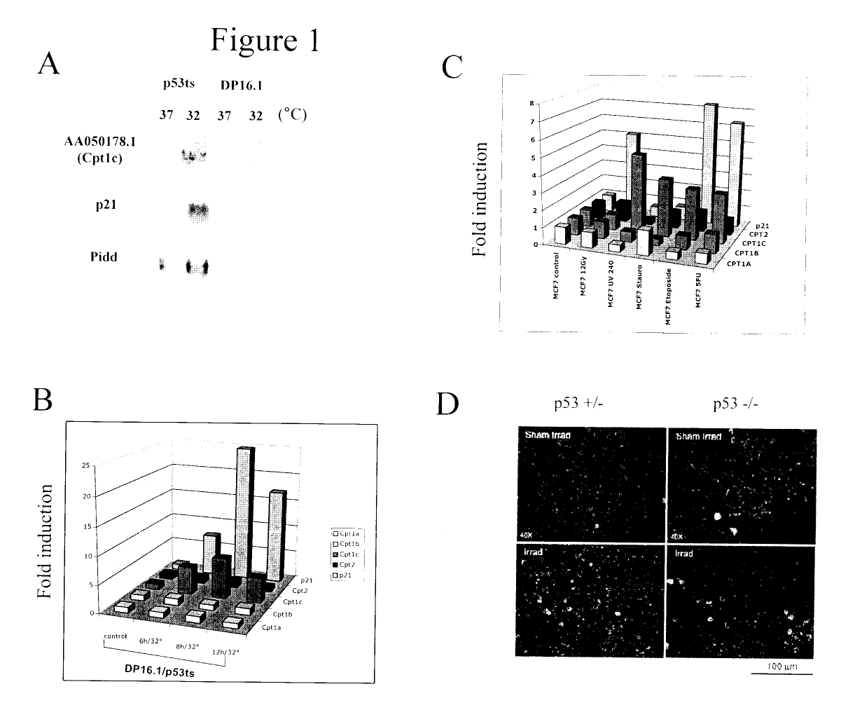

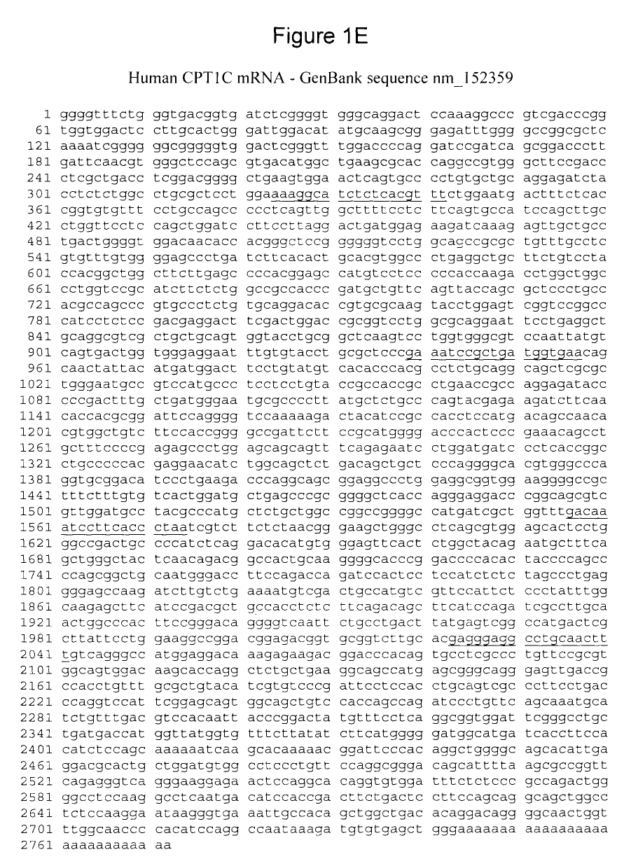

CPT1C is a p53 target gene—A cDNA microarray screen for identifying p53 transcription targets is known {Tsuchihara, K. et al. Ckap2 regulates aneuploidy, cell cycling, and cell death in a p53-dependent manner. Cancer Res 65, 6685-91 (2005)}. Briefly, this screen employed Friend virus-transformed mouse erythroleukemia cells that lack endogenous p53 and express a temperature-sensitive form of p53 (DP16.1/p53ts cells). Culture of DP16.1/p53ts cells at the permissive temperature of 32° C. activates p53. When DP16.1/p53ts cells were cultured for 3 or 6 hrs at 32° C., mRNA levels for EST AA050178.1, which represents a partial cDNA for murine carnitine palmitoyltransferase 1c (CPT1C), were increased 1.9-fold and 2.8-fold, respectively, (data not shown). In contrast, no significant changes in CPT1C mRNA levels in the parental (p53-deficient) DP16.1 control cultures were observed. Confirmatory Northern blot analysis using the full-length Cpt1c ORF as a probe showed that Cpt1c mRNA was upregulated in DP16.1/p53ts cells in a temperature-dependent manner. See FIG. 1(A). Densitometry of this Northern blot revealed a 4-fold induction of Cpt1c mRNA in temperature-shifted DP16.1/p53ts cells but no Cpt1c induction in temperature-shifted DP16.1 cells. As also shown in FIG. 1(A), the levels of mRNA'derived from the known p53 target genes p21 and Pidd were also upregulated in DP16.1/ts53 cells but not in DP16.1 cells, confirming p53 activation in the former.

Real time RT-PCR was used to study the kinetics of Cpt1c induction in temperature-shifted DP16.1/p53ts cells. As shown in FIG. 1(B), Cpt1c mRNA reached a maximal 5-fold induction after 8 hrs at 32° C., a pattern similar to that of the positive control p21 mRNA. Moreover, as shown in FIG. 1(C), in response to various stress stimuli (UV radiation, staurosporine (a broad spectrum inhibitor of protein kinases) or chemotherapy agents, etoposide and 5-fluorouracil) known to activate p53, CPT1C was upregulated in a variety of human and murine cell lines in a p53-dependent manner. Similar results were also obtained for brain (U87) and lung cancer cell lines. As shown in both FIGS. 1(B) and 1(C), CPT1C was the only CPT family member regulated by p53. CPT1C was thus selectively upregulated with respect to CPT1A and CPT1B.

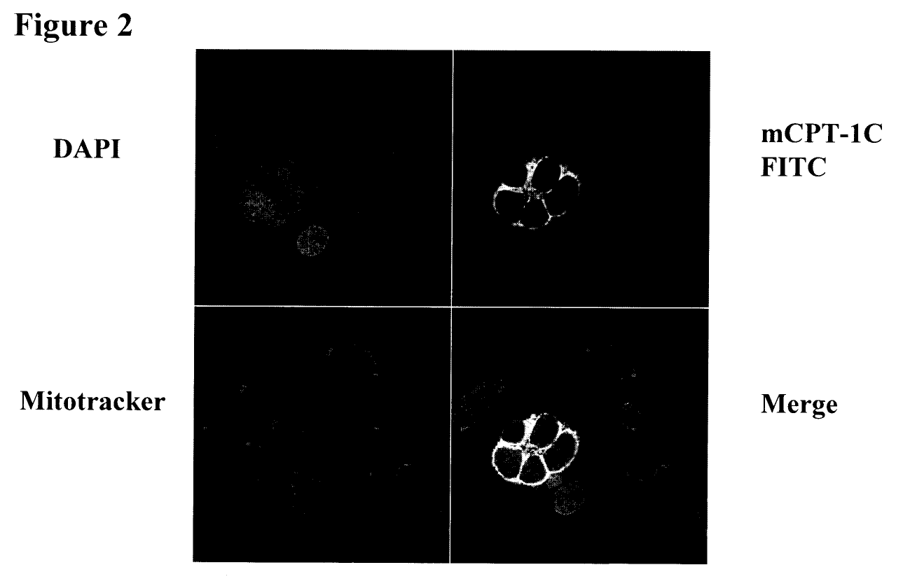

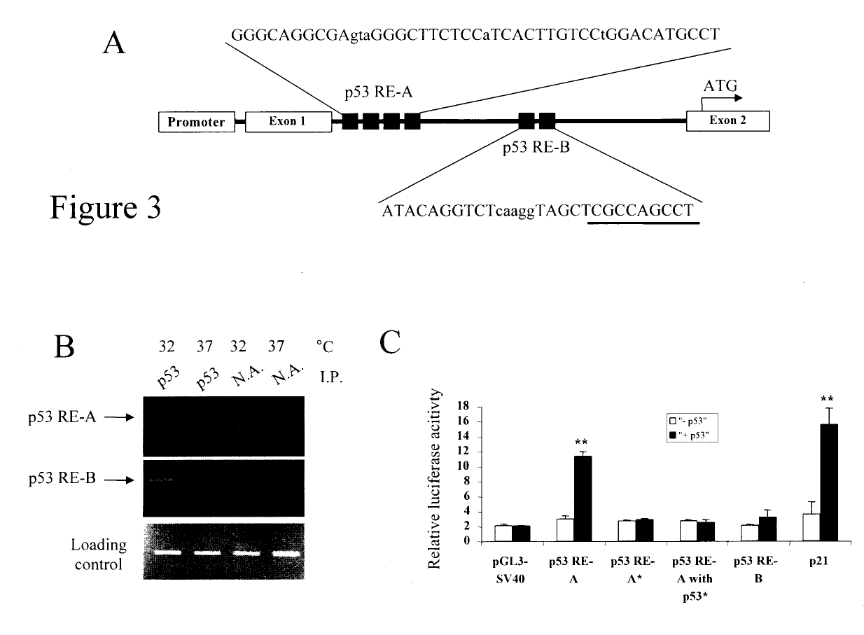

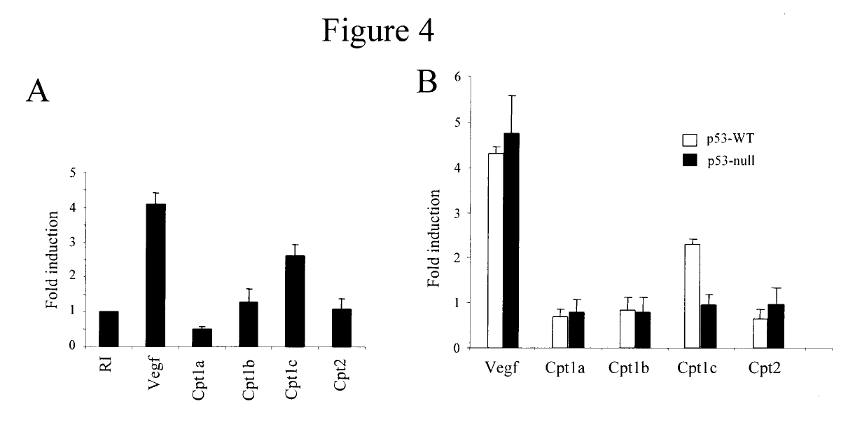

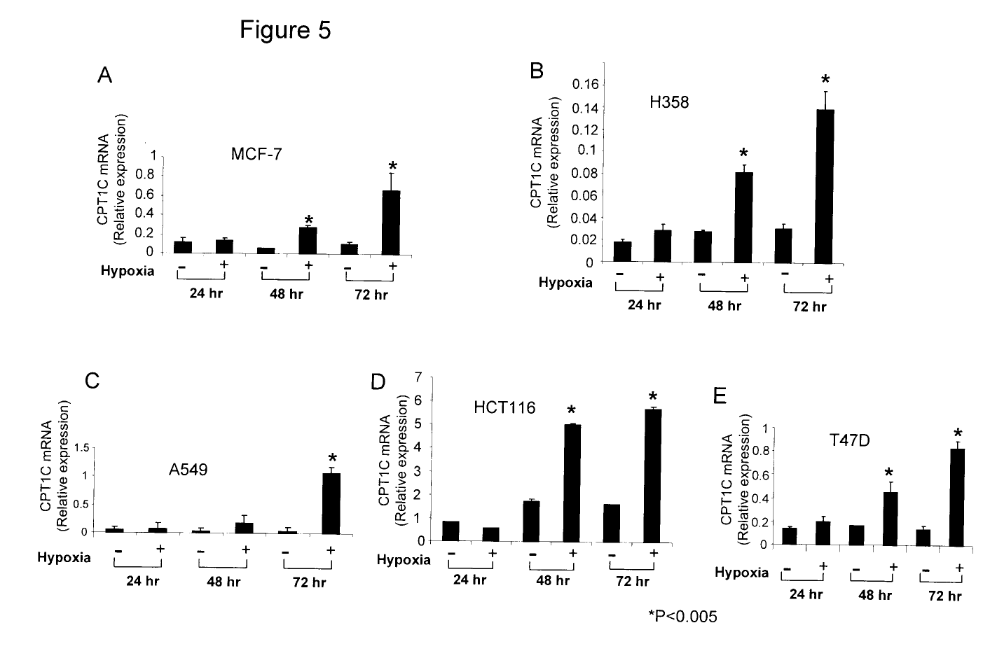

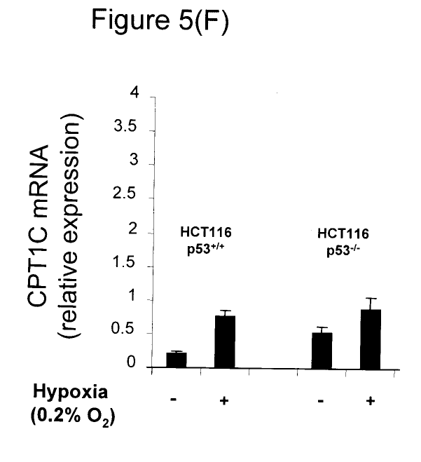

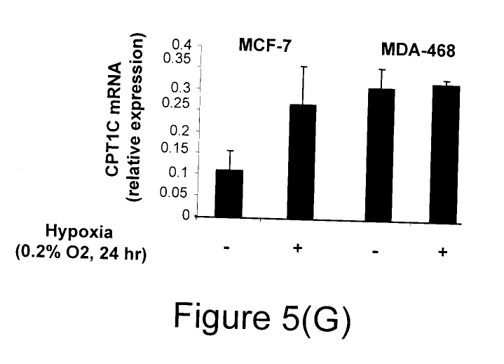

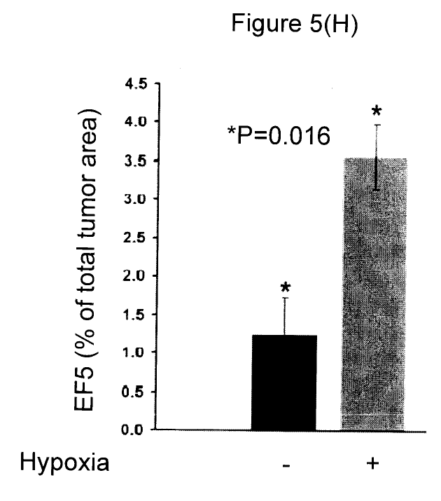

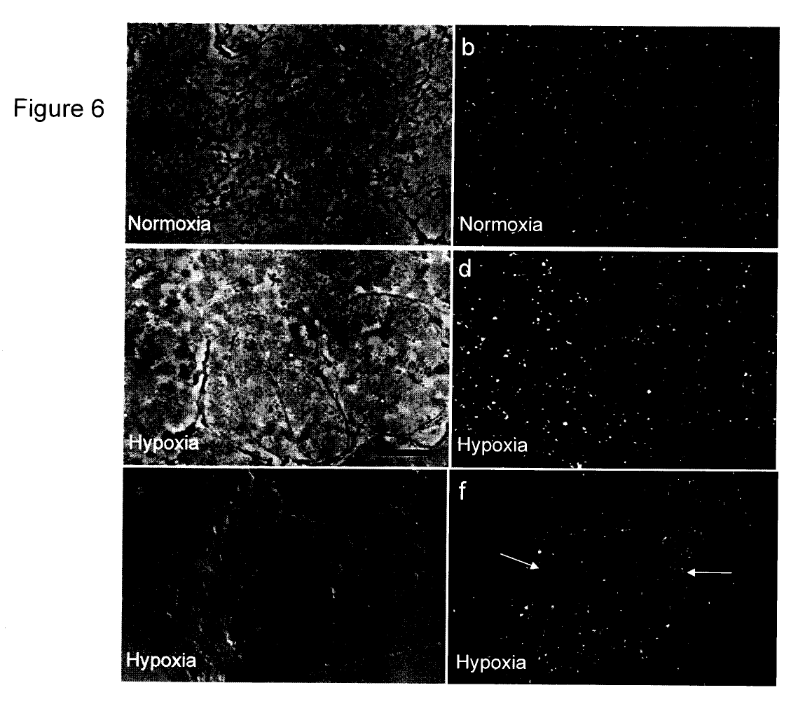

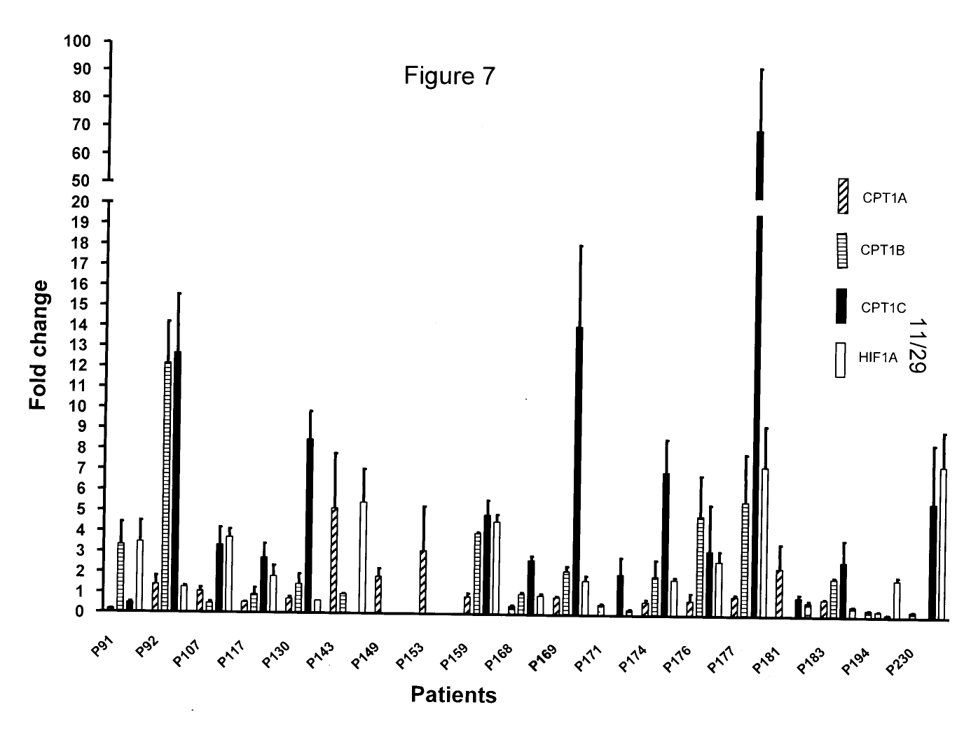

p53 upregulates CPT1C in vivo—To determine whether Cpt1c is upregulated in response to p53 activation in vivo, Cpt1c mRNA levels in irradiated mouse embryos were examined. At day 12.5 post-coitum, embryos of C57/b16 p53+/− and p53−/− mice were subjected in utero to 5 Gy irradiation. At 8 hrs post-irradiation, various tissues were excised and fixed for detection of Cpt1c mRNA by in situ hybridization. Consistent with previous reports, the highest base levels of Cpt1c mRNA were detected in the neuronal tissues of non-irradiated embryos. However, as can be seen by comparing panels A (upper left) and C (lower left) of FIG. 1(D), irradiated p53+/− embryos showed strong upregulation of Cpt1c mRNA in most tissues examined, including in the midbrain. A comparison of panels B (upper right) and D (lower right) of FIG. 1(D), however, shows that this Cpt1c upregulation was not seen in irradiated p53−/− embryos. The detection of p21 mRNA upregulation in irradiated p53+/− embryos confirmed that p53 had been activated. These results thus indicate that Cpt1c expression can be transcriptionally activated by p53 in vivo in response to DNA-damaging stimuli. Overexpression of a FLAG-tagged version of CPT1C in HeLa cells was executed and confocal microscopy performed. As shown in FIG. 2, analysis with DAPI and Mitotracker staining showed that CPT1C, like other CPT family members, is localized in the mitochondria. Northern blot analysis also confirmed that CPT1C mRNA is mainly expressed as a single transcript in brain and testes.
p53 directly activates CPT1C transcription—The nucleotide sequence of the murine Cpt1c promoter region was analyzed and two putative p53-responsive elements (p53-RE) were identified in the first intron. As illustrated in FIG. 3(A), these elements are 330 by apart: p53-RE-A, +174-219; p53-RE-B, +504-533. To investigate whether p53 could bind directly to these sites, ChIP analyses were performed on DP16.1/p53ts cells grown at 37° C. or 32° C. for 8 hrs. As shown in FIG. 3(B), using immunoprecipitation with anti-p53 antibody and PCR with primers specific for the two potential p53-binding sites, a strong amplification of p53-RE-A but not p53-RE-B was observed. To test the transcriptional activity of these sites, the p53-RE-A and p53-RE-B sequences were cloned into separate luciferase reporter vectors and these were cotransfected, along with either wild type (WT) p53 or p53 bearing a mutation in its DNA binding domain, into p53−/− mouse embryonic fibroblasts (MEFs). As can be seen in FIG. 3(C), increased luciferase activity was observed only when the p53-RE-A reporter was cotransfected with WT p53, demonstrating that transcription driven by p53-RE-A depends on p53's DNA binding activity. Furthermore, as shown in FIG. 3(C), a point mutation (G→T) at position 42 of p53-RE-A blocked p53-dependent luciferase activity. Taken together, these data demonstrate that p53-RE-A is both sufficient and necessary to drive p53-dependent transcription of CPT1C.
CPT1C expression is induced by hypoxia in a p53-dependent manner—p53 is known to be mutated in over 50% of all solid tumors and cancers are often associated with hypoxia. Induction of Cpt1c in hypoxic murine embryonic stem (ES) cells was thus studied. As indicated by FIG. 4(A), when the ES cells were cultured in 0.2% O₂ for 24 hrs and RNA expression patterns were determined using real-time RT-PCR, it was found that Cpt1c was the only CPT family member that was upregulated in response to hypoxia. To determine if this upregulation was dependent on p53, MEFs expressing either WT or mutant p53 were subjected to 0.2% hypoxia for 7 hrs and measured the mRNA expression of all CPT family members. As can be seen in FIG. 4(B), Cpt1c was the only CPT family member to be upregulated in a p53-dependent manner during hypoxia. To assess whether CPT1C was similarly upregulated in human tumor cells under hypoxic conditions, various human cancer cell lines were subjected to 0.2% hypoxia for 24, 48 or 72 hrs. As shown in FIGS. 5(A) to 5(E), CPT1C mRNA was upregulated in response to hypoxia. As indicated by FIG. 5(F), however, hypoxia-induced expression of CPT1C in human tumor cells does not seem to depend entirely on a functional p53 since induction of the CPT1C expression was also observed in HCT116 p53 null cells, albeit to a lesser degree. To demonstrate that CPT1C expression is induced in tumor cells in vivo by hypoxic conditions, tumor-bearing PyMT transgenic mice were subjected to a chronic hypoxic condition. The tumor tissues from both hypoxia-treated and control mice were then analyzed for EF5, a known hypoxia marker {H. W. Salmon, D. W. Siemann, Radiother. Oncol. 73, 359 (2004)} and Cpt1c expression. As shown in FIG. 5(H), the chronic hypoxia-treated tumor tissues were indeed more hypoxic than the untreated tumors as evidenced by a significant increase in the staining of a hypoxia marker, EF5. As seen in panel b of FIG. 6, low but significant levels of Cpt1c expression was detected in the tumor tissues. As indicated by panel d of FIG. 6, the tumor expression of Cpt1c was substantially increased when the mice were treated with chronic hypoxia. Panel f of FIG. 6 shows that the up-regulation of Cpt1c by hypoxia is mainly restricted to the tumor cells. These data show that CPT1C is induced under conditions of metabolic stress, especially hypoxia in tumor cells in both in vitro and in vivo and suggest that this molecule may be an important mediator of cancer cell survival.
CPT1C is upregulated in most human lung cancer samples—Because Cpt1c expression is present and induced in murine tumor cells by hypoxia, it was thought that the expression of CPT1C might be naturally upregulated in human tumors. Real-time RT-PCR was used to determine the levels of CPT1C mRNA in paired lung tumor and normal tissue samples from 19 patients with non-small cell lung carcinoma (NSCLC). As shown in FIG. 7, compared to matched normal control samples, higher levels of CPT1C mRNA were present in 13 of 19, 68% of, lung tumor samples tested. Because p53 is known to be frequently inactivated in NSCLC, p53 status was examined by immunohistochemistry staining to see whether there is a correlation between p53 status and CPT1C expression. Although all five p53-positive tumors (P117, P130, P159, P169 and P177) showed up-regulated CPT1C expression, CPT1C expression was also observed in several p53-negative tumors analyzed (P92, P107, P168, P171, P174, P183). See Table I. This is consistent with the notion that CPT1C expression in tumors may be not regulated entirely in a p53-dependent manner.

TABLE I
Status in the Human Lung Tumors

| Tumor | p53 Status |
|---|---|
| P91 | 0 |
| P92 | 0 |
| P107 | 0 |
| P117 | 1 |
| P130 | 1 |
| P143 | 0 |
| P149 | 0 |
| P153 | NA |
| P159 | 1 |
| P168 | 0 |
| P169 | 1 |
| P171 | 0 |
| P174 | 0 |
| P176 | NA |
| P177 | 1 |
| P181 | 0 |
| P183 | 0 |
| P194 | NA |
| P230 | NA |

p53 status was determined using standard immunohistochemistry staining method. “1”, p53 positive; “0”, p53 negative; NA, not available.

The expression in the cell lines derived from breast, lung and prostate cancers was examined using real time RT-PCR analysis. As shown in FIGS. 8, 9 and 10, the brain-specific CPT1C was upregulated in approximately 50% of the cell lines tested compared to their corresponding normal control cells, but no significant increase of CPT1A or 1B was observed. Consistent with the previous reports, little CPT1C expression was observed in the normal cells. Expression of CPT1C was found to be significantly induced in all the cancer cell lines tested under hypoxia, that is, not only in the cell lines that has increased level of CPT1C (H358, FIG. 8(B), FIG. 5(B)) but also in those that do not show increased levels of CPT1C (MCF-7, T47D, A549 and HCT116) compared to the normal control cells (FIGS. 8(A) and 8(B), 5(A), 5(C), 5(D) and 5(E)). In contrast, the expression of CPT1A or CPT1B was not induced except that CPT1B mRNA was increased in HCT116 cell upon exposure to hypoxia (FIG. 10(B)).

Depletion of CPT1C in human cancer cell lines subjected to metabolic stress results in decreased cell proliferation and its role in fatty acid oxidation and ATP generation—Induction of apoptosis by depletion of CPT1C in hypoxic tumor cells was studied. Initially, four siRNAs targeting four regions of the CPT1C mRNA sequence were transfected individually into the human breast cancer cell line MCF-7. Using quantitative RT-PCR, it was confirmed that a ˜70-80% reduction in CPT1C mRNA in the treated cancer cell lines compared to cells treated with a control siRNA targeting luciferase gene expression, the results being shown in FIG. 11(A). siRNAs 1 and 2, which showed the highest knockdown efficiency, were used for subsequent experiments in MCF-7 cells, the breast cancer cell line Hs578T, and the colon cancer cell line HCT116. The siRNA-treated cells were cultured under 0.2% hypoxia for 1, 2 or 3 days and the proliferation of CPT1C siRNA-treated cultures against that of controls was measured. As shown in FIGS. 11(B) to 11(D), the depletion of CPT1C mRNA in hypoxic human cancer cell lines led to a statistically significant decrease in culture growth. Interestingly, the results shown in FIGS. 11(B) and 11(C) indicate that this decrease in the proliferation of MCF-7 and Hs578T cells appeared to be hypoxia-dependent.

The current invention is thus based in part on the discovery that reducing the effective amount of CPT1C in cells can lead to increased apoptosis, an effect that is particularly pronounced in tumor cells that are rapidly dividing to the point where hypoxic conditions have developed locally in patient tissue. Agents for blocking the CPT1C pathway, thereby inhibiting tumor growth and providing a medical treatment for tumors and cancer have been developed. In particular, the inventionprovides siRNA compounds and methods for inhibition of the expression of CPT1C.

One aspect of the present invention provides a method for reducing the growth rate of a tumor expressing CPT1C. Such method comprises administering an amount of a nucleic acid therapeutic agent that inhibits gene expression of CPT1C.

To see whether CPT1C might have a role in fatty acid oxidation similar to other members of the CPT1 family, a control vector or vector expressing FLAG-tagged CPT1C was constitutively expressed in MCF-7 cells and fatty acid oxidation (FAO) measured using ¹⁴C-palmitic acid as a substrate. {X. Wang et al., Assay and Drug Development Technologies 2, 63 (2004)} The expression of FLAG-CPT1C was confirmed by Western blotting. The results, shown in FIG. 11(E), indicate that the FAO was increased by 356% in the CPT1C-expressing cells compared to the vector-only control cells. As indicated in FIG. 11(F), the ATP level was significantly reduced in the CPT1C-depleted PC3 cells in a time-dependent manner, suggesting that depletion of CPT1C expression accelerates ATP depletion in these cells. A similar but more pronounced effect was seen with CPT1A, a known FAO regulator. This effect was observed only in the absence of glucose. These findings suggest that CPT1C may play a role in fatty acid oxidation and inhibition of CPT1C activity in cancer cells may lead to reduced FAO and subsequently decrease in ATP production, particularly when glucose is limiting.

Effects of a reduction in CPT1C on the proliferation of human cancer cells subjected to glucose deprivation were then examined. MCF-7 cells and the human lung cancer line A549 were transfected with either CPT1C siRNA or control siRNA and cultured in 20 mM glucose to saturate glucose metabolism. At 24 hrs post-transfection, various concentrations of the glycolytic inhibitor 2-deoxy-D-glucose (2-DG), a metabolically inert form of glucose, were added to each culture. Cell proliferation was determined 5 days later using sulforhodamine B staining. As shown in FIG. 11(H), it was that, compared to A549 cells transfected with control siRNA, A549 cells transfected with CPT1C siRNA showed decreased proliferation in the presence of increasing concentrations of 2-DG. Similar results were obtained for MCF-7 cells, as shown in FIG. 11(I). These data suggest that modulating the level of CPT1C expression may render tumor cells sensitive to glycolytic inhibitors.

A second aspect of the invention is thus based on the discovery that the antitumor effect of reducing the effective amount of CPT1C in cells can be augmented by inhibiting glycolysis of the cells. A particular embodiment of this aspect of the invention thus provides a method for reducing the growth rate of a tumor expressing CPT1C by administering an amount of a nucleic acid therapeutic agent that inhibits gene expression of CPT1C and by administering a glycolysis inhibitor.

Loss of CPT1C function leads to spontaneous mitochondrial apoptosis—A murine ES cell line (clone XL823; BayGenomics), previously shown to be heterozygous for the gene-trap (gt) vector insertion into intron 6 of the Cpt1c gene shown in FIG. 12(A), was used to study protective functions of CPT1C. The gt mutation prematurely terminates Cpt1c transcription. RT-PCR and Southern blot experiments were conducted to confirm that there was a single gt insertion in the genome of XL823 cells. XL823 cells were then selected in a high concentration of G418 to generate ES cells homozygous for the gt mutation. As indicated by the Southern blot of FIG. 12(B), the presence of both Cpt1c^{gt/gt} and Cpt1c^{+/gt} cells in the treated culture was confirmed. To verify the Cpt1c deficiency in Cpt1c^{gt/gt} cells, real time RT-PCR using primers specific for sequences within exons 3, 7 or 9 of the Cpt1c gene was carried out. Cpt1c^{gt/gt} cells were found to be hypomorphic but retained 1% of normal expression levels of full-length Cpt1c mRNA, as indicated by FIG. 12(C). Despite their apparently normal viability, cultures of Cpt1c^{gt/gt} cells showed a greater than 40% decrease in total cell number after 2-3 days growth, compared to cultures of Cpt1c^{+/gt} cells, as shown in FIG. 13(A). A decrease in the mean diameter of the mutant cells was also observed, as seen in FIG. 13(B). As shown in FIG. 13(C), Annexin V staining of cells cultured under normal growth conditions revealed a modest (˜10%) but statistically significant increase in the rate of spontaneous apoptosis of Cpt1c^{gt/gt} cells compared to Cpt1c^{+/gt} cells. Consistent with this finding, a decrease in mitochondrial membrane potential and activation of caspases 3 and 9 were also observed in Cpt1c^{gt/gt} cells, as shown in FIG. 14. These results confirm the findings made using siRNA knockdown, demonstrating that CPT1C deficiency increases apoptosis.
Cpt1c^{gt/gt} cells are characterized by enlarged mitochondria and lipid droplets and defect in fatty acid homeostasis—Electron microscopy of Cpt1c^{gt/gt} cells revealed the presence of swollen mitochondria exhibiting a highly abnormal internal membrane structure and a loss of internal cristae density. The mutant mitochondria also contained numerous small vesicles not found in the mitochondria of Cpt1c^{+/gt} cells. As seen in FIG. 15, the cytoplasm of Cpt1c^{gt/gt} cells showed an accumulation of lipid droplets that was not present in either Cpt1c^{+/gt} cells or WT ES cells, suggesting that Cpt1c deficiency in the ES cells leads to a defective homeostasis of fatty acids. To identify what fatty acid species are affected, the fatty acids in the ES cells were profiled. AS summarized in Table II, relative amounts of several major fatty acids were dramatically altered in the Cpt1c deficient ES cells in comparison to the WT controls. For example, palmitoleic acid (C16:1), linoleic acid (C18:2 N6), arachidonic acid (C20:4 N6), and docosatetraenoic acid (C22:4 N6) were substantially increased in the Cpt1c deficient ES cells while oleic acid (C18:1) and gadoleic acid (C20:1) along with several other minor fatty acids were reduced. Since the Cpt1c cells showed abnormal mitochondrial membrane structures, examined the phosphoglycerides, the major components of the cell membranes, in particular phosphatidylcholine and phosphatidylethanolamine, two most abundant phosphoglycerides were also examined. Consistent with the findings above, the results summarized in Table III show that the corresponding phosphatidylcholines and phosphatidylethanolamines: C18:2 N6, C20:4 N6 and C22:4 N6 were substantially increased while C18:1 and C20:1 species reduced in the Cpt1c-deficient ES cells. Thus, CPT1C may be involved in lipid homeostasis and thus affect cell survival and growth through maintenance of mitochondrial membrane stability and lipid metabolism.

TABLE II
Fatty Acid Analysis in the Cpt1c WT
and Cpt1c gt/gt Murine ES Cells

| | | ES cells- | ES cells- |
|---|---|---|---|
| Fatty Acid | Name | Cpt1c wt | Cpt1c gt/gt |
| C8:0 | Caprylic acid | 0.00 | 0.00 |
| C10:0 | Capric acid | 0.00 | 0.00 |
| C12:0 | Lauric acid | 0.00 | 0.00 |
| C14:0 | Myristic acid | 1.24 | 1.62 |
| C14:1 | Myristoleic acid | 0.38 | 0.65 |
| C15:0 | Pentadecanoic acid | 0.34 | 0.56 |
| C16:0 | Palmitic acid | 13.11 | 14.36 |
| C16:1 | Palmitoleic acid | 1.78 | 2.33 |
| C18:0 | Stearic acid | 20.53 | 19.56 |
| C18:1 | Oleic acid | 33.25 | 24.30 |
| C18:2N6 | Linoleic acid | 2.82 | 4.80 |
| C18:3N6 | gamma linolenic acid | 0.00 | 0.00 |
| C18:3N3 | alpha linolenic acid | 0.27 | 0.00 |
| C18:4N3 | Parinaric acid | 0.25 | 0.65 |
| C20:0 | Arachidic acid | 0.70 | 0.16 |
| C20:1 | Gadoleic acid | 1.60 | 0.78 |
| C20:2N6 | Eicosadienoic acid | 0.20 | 0.79 |
| C20:3N6 | Eicosatrienoic acid | 2.26 | 2.32 |
| C20:4N6 | Arachidonic acid | 9.72 | 14.57 |
| C20:3N3 | Eicosatrienoic acid | 0.09 | 0.00 |
| C20:4N3 | Eicosatetraenoic acid | 0.00 | 0.00 |
| C20:5N3 (EPA) | Eicosapentaenoic acid | 0.67 | 0.00 |
| C22:0 | Behenic acid | 0.49 | 0.00 |
| C22:1 | Docosenoic acid | 0.54 | 0.00 |
| C22:2N6 | Docosadienoic acid | 0.00 | 0.00 |
| C22:4N6 | Docosatetraenoic acid | 1.46 | 5.75 |
| C22:5N6 | Docosapentaenoic acid n-6 | 0.00 | 0.00 |
| C22:5N3 (DPA) | Docosapentaenoic acid n-3 | 3.22 | 2.64 |
| C22:6N3 (DHA) | Docosahexaenoic acid | 4.64 | 3.66 |
| C24:0 | Lignoceric acid | 0.43 | 0.00 |
| C24:1 | Nervonic acid | 0.00 | 0.47 |
| Total | | 100.00 | 100.00 |

The Fatty acids were analyzed using a standard method as a fee-for-service at the Lipid Laboratories (Guelph, Onatrio, Canada).

TABLE III
Phosphoglyceride Analysis of the Cpt1c wt and gtgt ES cells

| | Phosphatidyl | Phosphatidyl | Phosphatidyl | Phosphatidyl |
|---|---|---|---|---|
| | Choline | Choline | Ethanolamine | Ethanolamine |
| | ES cells- | ES cells- | ES cells- | ES cells- |
| FA Chain | Cpt1c wt | Cpt1c gt/gt | Cpt1c wt | Cpt1c gt/gt |
| C14:0 | 2.91 | 2.85 | 0.51 | 0.97 |
| C14:1 | 0.13 | 0.14 | 1.24 | 0.95 |
| C15:0 | 0.4 | 0.93 | 0.45 | 0.53 |
| C16:0 | 27.65 | 27.93 | 6.28 | 7.71 |
| C16:1 | 3.8 | 2.92 | 1.63 | 1.04 |
| C18:0 | 11.72 | 14.13 | 13.6 | 16.36 |
| C18:1 | 40.13 | 28.14 | 26.28 | 18.06 |
| C18:2N6 | 2.34 | 4.76 | 1.69 | 2.46 |
| C18:3N6 | 0.03 | 0.08 | 0.05 | 0.04 |
| C18:3N3 | 0.12 | 0.24 | 0.16 | 0.05 |
| C18:4N3 | 0 | 0.11 | 0.23 | 0.46 |
| C20:0 | 0.06 | 0.01 | 0.08 | 0.07 |
| C20:1 | 2.33 | 1.35 | 2.69 | 1.5 |
| C20:2N6 | 0.28 | 0.47 | 0.03 | 0.23 |
| C20:3N6 | 1.32 | 1.39 | 1.76 | 0.99 |
| C20:4N6 | 2.9 | 7.73 | 17.72 | 21.71 |
| C20:3N3 | 0.02 | 0.08 | 0.04 | 0.04 |
| C20:4N3 | 0.06 | 0.05 | 0.1 | 0.08 |
| C20:5N3 | 0.21 | 0.34 | 1.4 | 0.64 |
| C22:0 | 0.02 | 0.05 | 0.15 | 0.1 |
| C22:1 | 0.24 | 0.4 | 1.11 | 0.63 |
| C22:2N6 | 0.02 | 0.16 | 0.87 | 0.46 |
| C22:4N6 | 0.6 | 1.95 | 3.49 | 8.12 |
| C22:5N6 | 0.05 | 0.3 | 0.21 | 0.68 |
| C22:5N3 | 0.98 | 1.66 | 6.53 | 6.33 |
| C22:6N3 | 1.34 | 1.78 | 11.64 | 9.46 |
| C24:0 | 0.23 | 0.05 | 0.04 | 0.22 |
| C24:1 | 0.08 | 0 | 0.03 | 0.11 |
| Total | 100 | 100 | 100 | 100 |

The phosphoglycerides were analyzed using a standard method as a fee-for-service at the Lipid Laboratories (Guelph, Ontario, Canada).

CPT1C protects ES cells from hypoxia-induced cell death—DNA damaging agents including ionizing radiation, UV, and cisplatin demonstrated no significant change in cell viability between the CPT1C heterozygous and hypomorphic ES cells (data not shown). As shown in FIG. 16(A), when these cells were treated for 24 hours under hypoxic conditions (0.2% oxygen) approximately 80% of the hypomorphic ES cells were undergoing apoptotic cell death, whereas cell death in the heterozygous ES cells was only 7% indicating that CPT1C protects cells from hypoxia-induced cell death.

Hypoxic growth is also known to enhance acidosis of cultured cells. To exclude that acidosis is the main stress inducer, cells were treated with hypoxia in the presence of either 25 mM or 100 mM HEPES. Additional buffering of the media with the added HEPES could not recover cell death in the hypomorphic ES cells suggesting that reduced oxygen and not apoptosis is the primary stress that induces cell death in the CPT1C-deficient cells. Since reduced ATP production would be a consequence of mitochondrial dysfunction accentuated under hypoxic conditions, we tested whether reduced glucose would further reduce cell viability in the CPT1C-deficient cells. Withdrawal of glucose from the media during hypoxia increased cell death in the homozygous CPT1C-deficient cells (hypomorph 80%, heterozygous 60%, and wild-type 10%). Under conditions of glucose withdrawal and hypoxia, the heterozygous ES cells also demonstrated a reduced viability compared to wild-type ES cells, as shown in FIGS. 16(B) and 16(C), indicating that the CPT1C gene trap allele has a haplo-insufficient phenotype under conditions that do not support glycolysis which compensates for reduced mitochondrial function.

To parallel experiments in CPT1C siRNA-treated tumor cells, the responses of Cpt1c^{+/gt} and Cpt1c^{gt/gt} cells to hypoxia and glucose deprivation were examined. It was first confirmed that Cpt1c^{+/gt} and Cpt1c^{gt/gt} cells had normal p53 function in that no significant differences in viability were observed when the cells were subjected to DNA-damaging agents such as ionizing radiation, UV or cisplatin. However, as seen in FIG. 16(D), when cultured for 24 hrs under hypoxia, 79% of Cpt1c^{gt/gt} cells underwent apoptotic cell death, whereas only 11% of Cpt1c^{+/gt} cells did so. Because growth under hypoxic conditions is known to enhance the acidosis of cultured cells, and acidosis is a known stress factor, the culture medium of the hypoxic cells was buffered with either 25 mM or 100 mM HEPES. However, as shown in FIG. 16(E), the addition of HEPES did not prevent the excessive death of hypoxic Cpt1c^{gt/gt} cells. Thus, it is the reduction in oxygen and not the acidosis of hypoxic culture conditions that induces the death of CPT1C-deficient cells. Taken together, the data show that CPT1C protects multiple cell types from hypoxia-induced death.

The effect of glucose deprivation of the viability of Cpt1c^{gt/gt} cells was examined. As can be seen in FIG. 16(F), withdrawal of glucose from the culture medium resulted in the apoptotic death of 34% of Cpt1c^{gt/gt} cells and 17.8% of Cpt1c^{+/gt} cells but only 5% of control WT ES cells. These data confirm that CPT1C depletion renders cells prone to apoptotic death under conditions of metabolic stress.

Antitumor activity in vivo of inhibition of CPT1C activity—The above observations also indicate that inhibition of CPT1C activity may have antitumor activity in vivo. To directly test antitumor activity, CPT1C expression was deleted in the breast cancer MBA-MD-468 cells using retrovirally mediated shRNA against CPT1C or GFP as control. Theses cells were transplanted as xenografts into immune-deficient mice at either 1.5×10⁶ or 5×10⁶ cells per implant and the tumors monitored for approximately 10 weeks. As shown in FIG. 17, compared to the GFP control tumors, the CPT1C-depleted tumors were found to be significantly growth-suppressed. At the end of the 10 weeks, the average size of the CPT1C-depleted tumors was decreased by 64% and 57% in comparison to the control tumors, respectively.
Expression and preparation of CPT1A and CPT1C preparations—The sequence for CPT1A, given in C H Britton et al., Proc Natl Acad Sci USA. 1995 Mar. 14; 92(6): 1984-1988, and is specifically incorporated herein by reference. The sequence for CPT1B is given in Naoshi Yamazaki et al. Biochimica et Biophysica Acta (BBA)—Gene Structure and Expression, Volume 1307, Issue 2, 7 Jun. 1996, pages 157-161, and is specifically incorporated herein by reference. Nucleotide sequences encoding human CPT1 enzymes are individually cloned into the yeast expression vector pESC-trp at the Cla1 (5′ terminus) and Pac1 (3′ terminus) restriction sites by PCR amplification of the open reading frame using oligonucleotide primers designed to encode the wild-type CPT1 proteins. Standard molecular biology techniques are used to transform and express the CPT1 proteins in the yeast Saccharomyces cerevisiae. The yeast cells are lysed by enzymatic degradation of the cell wall by Zymolase, and the mitochondria are isolated by standard biochemical techniques. The integrity of the isolated mitochondria is monitored by determining the activity of succinate dehydrogenase in the mitochondrial preparations. The mitochondrial extracts were stored at −80° C. in buffer containing 10 mM HEPES pH 7.4 and 250 mM sucrose.

cDNAs encoding human CPT1 genes are also individually cloned into the pcDNA3.1 vector for expression in cultured mammalian cells. Cells expressing the exogenous CPT1 are identified and grown under standard conditions. Mammalian cells are harvested, and mitochondrial extracts prepared using standard biochemical methods. The mitochondrial extracts are stored at −80° C. in buffer containing 10 mM HEPES pH 7.4 and 250 mM sucrose.

Alternatively, an approach similar to that described by Sierra et al. {Adriana Y Sierra, Esther Gratacós, Patricia Carrasco, Josep Clotet, Jestis Ureiia, Dolors Serra, Guillermina Asins, Fausto G. Hegardt, Núria Casals. J. Biol. Chem., 10.1074/jbc.M707965200, published online Jan. 11, 2008} Sierra et al. cloned the coding region of rat cpt1a and cpt1c in vector pIRES2-EGFP and overexpressed the cpt1 proteins in PC-12 cells. Cells were incubated for 2 hours with [1-¹⁴C]palmitate and palmitate oxidation to CO₂ was measured. It is thus contemplated that a similar method can be used with the coding regions of human CPT1A and human CPT1C.

Other methods for assaying carnitine palmitoyl transferases are known in the literature. These methods either rely on the generation of a radiolabeled product, as Sierra et al. used, which can be quantified using a scintillation counter, or on a coupled colorimetric method which can be used to quantify residual substrate or generated product by spectrophotometric methods. A typical method used to generate and detect a radiolabeled product is described below as an excerpt from the reference Bremer et al., Biochimica et Biophysica Acta, 833 (1985), 9-16, and additional permutations of the radiolabel assay have been described by McGarry et al., Journal of Biological Chemistry 1978, 253; 4128. A typical method used to detect generated product using a colorimetric detection system is described below as an excerpt from the reference.

Generally speaking, known radiolabeled assays for the detection of palmitoylcarnitine utilizes the incorporation of a water soluble radiolabeled substrate carnitine into a less soluble product palmitoylcarnitine, which can be selectively extracted with an organic solvent such as n-butanol and quantified with a standard scintillation counter. An excerpt from the reference by Bremer et al., Biochimica et Biophysica Acta, 833 (1985), 9-16 reads as follows: “Carnitine palmitoyltransferase was assayed with a modification of the butanol extraction procedure (Norum, 1965, Biochim. Et Biophys. Acta 99, 511-522). The standard enzyme assay mixture contained in a volume of 0.5 mL 0.2 mM (−)-[methyl-³H]carnitine (approx. 10000 dpm/nmol), 50 uM palmitoyl-CoA/20 mM Hepes buffer (pH 7.0)/1 or 2% fatty acid free bovine serum albumin/40-75 mM KCl, and in most experiments 7.5 mM sucrose and 22.5 mM mannitol from the added suspension of mitochondria or mitochondrial membranes. With intact mitochondria 2 mM KCN was added to inhibit oxidation of the palmitoylcarnitine formed. All incubations were done at 30° C. for 2-5 min. Addition of malonyl-CoA and preincubation conditions were as stated in the different figures and tables. The reaction was stopped by addition of 2 mL 6% HClO₄. After centrifugation at 3000 rpm for 10 min the precipitate was washed once with 2 mL 6% HClO₄ and dissolved in 1.6 mL water and vortexed with 1 mL n-butanol. Subsequently, 0.4 mL of 6% HClO₄ and 0.1 mL saturated ammonium sulfate were added and the mixture was vortexed after each addition. Finally, after centrifugation to separate the water and n-butanol phases, 0.5 mL of the butanol phase was mixed with 10 mL of Brays scintillation fluid and counted in a Packard scintillation counter.

The reverse reaction was assayed by incubating approx. 50 uM [3H]palmitoylcarnitine, 100 uM CoA 20 mM Hepes (pH 7), 1% albumin, 50-80 mM KCl and mitochondrial membranes in a total volume of 0.5 mL for 5 min at 30° C. The reaction was stopped with 1 mL 6% HClO₄. After centrifugation 0.5 mL of the clear supernatant was neutralized with 1 M KOH, and the (−)-[methyl-3H]carnitine formed was measured by counting 0.5 mL of the neutralized supernatant in 10 mL scintillation fluid in a Packard scintillation counter.”

Generally speaking, known colorimetric assays for the detection of palmitoylcarnitine transferase activity utilizes the generation of a free CoA thiol followed by a chemical coupling of the free thiol to a colorimetric detection agent such as DTNB, also known as NBS₂ or Ellman's Reagent. The colorimetric product can be quantified with a spectrophotometer at 412 nm. An excerpt from the reference Saggerson E. D. Biochemical Journal 1982, 202; 397 is reproduced here: “In all cases, reactions were performed with 50 ul samples of mitochondria in a final volume of 1.0 ml, which contained 10 mM-Tris/HCl buffer, pH 7.4, and fatty acid-poor albumin (1.3 mg/ml). Included with these components were various proportions of sucrose and KCl such that together these always provided an osmolarity in the assay mixture of 300 mosmol/litre. Thus, when stated that [K+] was zero, [sucrose] was 300 mM or, for example, when [K+] was 40 mM, [sucrose] was 220 mM etc. The Nbs2-linked assay was performed in a Unicam SP. 8-100 spectrophotometer with 0.1 mM-Nbs2 present. The reaction was initiated by addition of 400 nmol of L-carnitine, which was omitted from blank cuvettes.”

The radiolabeled or colorimetric assays described above for monitoring the activity of CPT1A may be adapted for use in detecting CPT1C activity in a biochemical reaction under appropriate conditions, particularly as has been carried out by Sierra et al. The subject screening assays can be performed in the presence or absence of other agents.

Agents which act directly on CPT1C, and preferably act selectively on CPT1C with respect to CPT1A, CPT1B or both CPT1A and CPT1B, are screened using preparations containing CPT1C. As disclosed by Price et al., there is a high degree of sequence identity between the amino acid sequences of the members of the CPT1 protein family. Agents shown to be selective in their binding and/or inhibitory capacity for CPT1C over other members of the CPT1 family, particularly CPT1A or CPT1B, are thus more suitable as potential anticancer agents. Agents to be tested against CPT1C directly are preferably small molecules of the type known to modulate function of proteins with enzymatic function, and/or containing protein interaction domains.

Chemical agents, referred to in the art as “small molecule” compounds are typically organic, non-peptide molecules, having a molecular weight up to 10,000, preferably up to 5,000, more preferably up to 1,000, and most preferably up to 500 daltons. This class of modulators includes chemically synthesized molecules, for instance, compounds from combinatorial chemical libraries. Synthetic compounds may be rationally designed or identified based on structural information of ligand binding site, including substrates, in homology models constructed from 3D structures and sequences of proteins that are functionally or through sequence resemblance related to CPT1C and known or inferred properties of CPT1C or may be identified by screening compound libraries. Alternative appropriate modulators of this class are natural products, particularly secondary metabolites from organisms such as plants or fungi, which can also be identified by screening compound libraries for CPT1C-modulating activity. Methods for generating and obtaining compounds are known in the art (Schreiber S L, Science (2000) 151: 1964-1969).

An embodiment of this invention thus includes a method of screening anti-cancer agents for treating a human. The method includes (a) contacting a mammalian CPT1C protein with a test agent thought to be effective in inhibiting the activity of the CPT1C protein in the presence of a fatty acyl-CoA known to be a substrate of the CPT1C; (b) determining if the test agent inhibits the activity of the CPT1C, wherein determining if the test agent inhibits the activity of the CPT1C comprises quantitating the amount of fatty acyl-carnitine produced in the presence of the agent; and (c) classifying the test agent as a potential anti-cancer agent if the test agent inhibits the activity of the CPT1C. In a specific embodiment the known substrate is palmitoyl-CoA, which can be provided in the form of palmitic acid.

Once screened as potential anti-cancer agents, the agents can be used as lead compounds and tested more directly for effects on cancer cells, particularly cancer cells growing under hypoxic conditions, using the methods described herein. Promising anticancer agents can have their usefulness confirmed using in vivo models as described, for example, by Kiranmai Gumireddy et al., Cancer Cell, Volume 7, Issue 3, Pages 275-286, 2005. From the most promising lead compounds, candidate clinical compounds may be designed, optimized, and synthesized. The activity of candidate small molecule CPT1C-modulating agents may be improved several-fold through iterative secondary functional validation, structure determination, and candidate modulator modification and testing. Additionally, candidate clinical compounds are generated with specific regard to clinical and pharmacological properties. For example, the reagents may be derivatized and re-screened using in vitro and in vivo assays to optimize activity and minimize toxicity for pharmaceutical development.

### Examples

Cell lines—DP16.1 and DP16.1/p53ts cell lines were maintained in a-modified Eagle's medium (α-MEM) containing 10% fetal calf serum (FCS). Human cancer cell lines MCF7, Hs578T, A549, H358, PC3 and HCT116 and other cancer cells were purchased from ATCC and maintained according to the vendor's instructions. Normal human primary cells were purchased from Cambrex, Charles City, Iowa. p53+/+ and p53−/− mouse embryonic fibroblasts (MEFs) were derived from 14 day old embryos, transformed with E1A/Ras, and cultured in a 5% CO₂ atmosphere in Dulbecco's MEM containing 10% FCS. XL823, a gene trap ES cell line targeting Cpt1c (BayGenomics), was maintained on 1% gelatin-coated dishes in DMEM supplemented with leukemia inhibitory factor, 15% FCS, L-glutamine and β-mercaptoethanol.
CPT1C expression in cancer lines exposed to hypoxic conditions. Human cancer cell lines were purchased from ATCC and maintained and grown according to vendor's instructions. Human breast (MCF7, MDA468), lung (H358) and colon (HCT116) cancer were subjected to hypoxia conditions (2% in A and 0.2% in B) for various periods of time as indicated in FIG. 11. The cells were then harvested and total RNAs were prepared. The CPT1C mRNA levels were measured in a quantitative real time PCR assay using CPT1C-specific primers: (forward primer): 5′-GCC ATG GAG GAC AAA GAG AA (SEQ ID NO; 2) and (reverse primer) 5′-ACG ATG TAC AGC GCA AAC AG (SEQ ID NO: 3). The expression levels were normalized over that of GAPDH or beta-actin. The data were presented as fold increase (hypoxia vs normoxia).
Human lung tumor and normal samples—Matched tumor and normal lung tissue samples were harvested from 19 non-small cell lung carcinoma (NSCLC) patients treated by surgical resection without adjuvant chemotherapy at the University Health Network and Mount Sinai Hospital, Toronto, Canada. Tissues were harvested within 30 min after complete resection, and the quality and pathology of the tumor samples was confirmed by the study pathologist (M.-S.T.). The use of these human samples and their associated clinical information was approved by the Research Ethics Board of the University Health Network.

The p53 status was determined by a standard immunohistochemistry method. RNA was extracted from the NSCLC patient samples using phenol-chloroform {Chomczynski, P. & Sacchi, N.

CPT1C mRNA levels as well as levels of HIF1a were measured in the 19 paired lung tumor and matched normal tissues using gene-specific oligo primers: CPT1C forward primer: 5′-TGA CAT CCA CCG ACT TCT GAC T (SEQ ID NO: 4) and CPT1C reverse primer 5′-TGG CAA TTT CAC CCT TAT TCC T (SEQ ID NO: 5). Single-step method of RNA isolation by acid guanidinium thiocyanate-phenol-chloroform extraction. {Chomczynski, P. & Sacchi, N. Single-step method of RNA isolation by acid guanidinium thiocyanate-phenol-chloroform extraction. Anal Biochem 162, 156-9 (1987)} and purified using the RNeasy kit (Qiagen). Purified RNA was treated with DNase (Ambion) and quantified via spectrophotometry. RNA quality was assessed by agarose gel electrophoresis. Quantitative real-time PCR was performed using the SYBR Green assay and the ABI PRISM 7900-HT (Applied Biosystems). Each 10 μl quantitative RT-PCR reaction contained a 2 ng equivalent of cDNA in one well of a 384-well plate. Plates were incubated at 95° C. for 3 min followed by 40 cycles of 95° C. for 15 sec, 65° C. for 15 sec, and 72° C. for 20 sec. The data are presented as fold increase (tumor vs matched normal tissue). Fold changes in RNA expression were calculated from the average of duplicate samples with the delta-delta Ct method using β-actin as the housekeeping gene. Error bars represent the (average fold change)×(2SEM−1).

ChIP analysis—DP16.1 and DP16.1/p53ts cells were cultured at 37° C. or 32° C. for 6 hrs, cross-linked in formaldehyde and sonicated with 6×10 sec pulses at 50 watts, 50% max power (Vibra Cell™, Sonics and Material Inc). Extracts were subjected to ChIP assays using the Acetyl-Histone H3ChIP Assay Kit (Upstate Biotechnology, Charlottesville, Va.) and anti-mouse p53 antibody (FL-393; Santa Cruz). PCR amplification was performed using primers specific for the two regions in Cpt1c intron 1 that contained consensus p53-binding sequences. Un-precipitated genomic DNA was used as loading control.
Luciferase assay. The two potential p53 binding sites (RE-A: GGGCAGGCGAGTAGGGCTTCTCCATCACTTGTCCTGGACATGCCT (SEQ ID NO:6) and RE-B: ATACAGGTCTCAAGGTAGCTCGCCAGCCT (SEQ ID NO: 7) localized in the first intron of CPT1C were individually amplified by PCR from E14K embryo stem cells and cloned into a pGL3-promoter vector (Promega, Madison, Wis.). These constructs were transfected with Lipofectamine 2000 into p53^{-/-} mouse embryo fibroblasts. Luciferase activity was measured in the presence or absence of p53 and normalized to the simultaneous b-galactosidase. A luciferase construct containing the p21 promoter region, and a p53 construct with a mutation in the DNA binding site, were used as positive and negative controls, respectively.
Hypoxia induction of CPT1C in mouse embryonic stem cells (ES) and fibroblasts (MEFs)—Mouse ES cells heterozygous for CPT1C (wt/gt) were grown under 0.2% O₂ for 24 hrs The cells were then harvested and total RNA samples prepared for quantitative real time PCR analysis. mRNA levels of VEGF (a known hypoxia-induced gene), CPT1A, 1B and 1C were determined. The fold increase (hypoxia vs normoxia) was then calculated and presented.
Apoptosis induction, hypoxia and glucose withdrawal—MCF-7 cells were either sham-treated or treated with the following stress stimuli: 12 Gy γ-irradiation, 240 μJ/cm² UV, 1 μM staurosporine, 10 μM etoposide or 50 μg/ml 5-fluorouracil. For hypoxia, murine ES cells, MEFs or human MCF-7, H358, A549, HCT116 or Hs578T cancer cells were subjected to hypoxic conditions (0.2% O₂) in a hypoxia chamber (INVIVO2 400; BSBE Scientific). For glucose withdrawal, DMEM medium without glucose was used.
siRNA of CPT1C in cancer cell lines—MCF-7, A549, HCT116, H358 or Hs578T human cancer cells were seeded into 96-well plates at 1500-2500 cells per well depending on each cell line's growth rate. At 24 hrs post-seeding, cells were transfected with siRNAs using Lipofectamine 2000 (Invitrogen, Burlington, ON, CA). To determine CPT1C RNA knockdown efficiency, 4 siRNAs (Dharmacon, Lafayette, Colo., USA) were either individually transfected at 40 nM each, or as a pool of 10 nM each, into the seeded cells. Transfection of 40 nM of an siRNA targeting luciferase expression was used as the negative control (sicontrol). Quantitative RT-PCR was performed at 72 hrs post-transfection.

To determine the effect of CPT1C depletion on cell proliferation, 10 nM sicontrol or 10 nM of CPT1C siRNA1 or 2 was transfected into human cancer cells and incubated under normoxic or hypoxic conditions starting at 24 hrs post transfection. For normoxia, siRNA-transfected cells were incubated at 37° C. in 20% O₂ for 6 days. For hypoxia, siRNA-transfected cells were incubated in 0.2% O₂ for 1, 2 or 3 days before being transferred back to normoxia for 4, 3 or 2 days, respectively. Cell proliferation was determined using the SRB assay (see below). The sequences of the four individual CPT1C siRNAs used were: 1^{#}-5′-GAA AUC CGC UGA UGG UGA A (SEQ ID NO: 8); 2^{#}-5′-GAC AAA UCC UUC ACC CUA A (SEQ ID NO: 9); 3^{#}-5′-AAA GGC AUC UCU CAC GUU U (SEQ ID NO: 10); 4^{#}-5′-GAG GGA GGC CUG CAA CUU U (SEQ ID NO: 11), respectively. These sequences are underlined in SEQ ID NO: 1 shown in FIG. 1(E). Fatty acid oxidation and ATP assays—To examine FAO, MCF-7 cells (2×10⁶) were stably transfected with either control vector or an NH₂-terminally FLAG-tagged CPT1C expression construct and incubated for 1 hr at 37° C. in 5 ml Krebs Ringer buffer containing 5 mM glucose. The cells were washed with PBS and resuspended in 0.5 ml of Krebs Ringer Buffer, no glucose, 0.5% BSA, with 1 uCi of [1-¹⁴C] palmitic acid (GE Healthcare, NJ. USA) but no glucose. The cells were seeded in the centre well of a organ culture dish (Falcon 353037) and sealed with vacuum grease around the inner edge of the lid. The dishes where placed in a tissue culture incubator 37° C. at 5% CO₂ for 4 hours. After the incubation period the dishes were removed from the incubator, 1 ml of 1M NaOH was pipetted through a hole in the lid into the outer ring and 300 ul of 2N HCl was pipetted through a hole in the lid into the centre well containing the cells. The holes in the lid were resealed with Scotch tape and the release of radioactive labeled CO₂ from the media in the centre well was collected at room temperature over night. The following morning, 800 μl NaOH from the outer ring of the dish was added to 5 ml Ecoscint to determine cpm attributable to ¹⁴CO₂ production.

For ATP assay, PC-3 cells were cultured in low glucose (5.6 mM) DMEM medium containing 10% FBS and seeded in white 96-well tissue culture plates at 7000 cells/well. After 24 hours incubation, 40 nM of sicontrol (non-silencing control), CPT1C siRNA or CPT1A siRNA (Dharmacon, Lafayette, Colo., USA) were transfected into cells using Lipofectamine-2000 (Invitrogen, Burlington, ON, CA). 48 hours post transfection, culture medium were changed to 100 ul of PBS. Cellular ATP levels were then measured from time 0 to 24 hours. The ATP level was determined using the CellTiter-Glo Luminescent Cell Viability Assay (Promega, Madison, Wis.). Luminescence intensity from each well was measured using SepctraMax M5 (Molecular Devices, Sunnyvale, Calif.).

Real time PCR. Cell lines have been treated with different stress stimuli and RNA was extracted using the Qiagen Mini Kit (Sigma). RNA was reverse transcribed by Superscript (Invitrogen). Specific primers for mouse GAPDH, CPT1A, CPT1B, CPT1C, CPT2, p21, and Hif1a (positive control for induction by hypoxia) were designed using either Oligo 5 or PrimerBank. The PCR primers for the genes are as follows: mCPT1A, forward primer 5′-GAA CAT CGT GAG TGG CGT CCT C (SEQ ID NO: 12), reverse primer 5′-TCG ACC CGA GAA GAC CTT GAC C (SEQ ID NO: 13); mCPT1B; forward primer 5′-TGC ACA GCA AGA CCA GCC AT (SEQ ID NO: 14), reverse primer 5′-TTC CTT GGC CAA TGT CTC CA (SEQ ID NO: 15); mCPT1C; forward primer 5′-CAC CCT TCA TGT GGC TCT GAG (SEQ ID NO: 16), reverse primer 5′-GGT GCC TCC CGG AAA AGA T (SEQ ID NO: 17); mVEGF; forward primer 5′-TAC TGC CGT CCG ATT GAG AC (SEQ ID NO: 18), reverse primer 5′-TGA TCT GCA TGG TGA TGT TG (SEQ ID NO: 19); RNAse inhibitor, forward primer 5′-TCC AGT GTG AGC AGC TGA G (SEQ ID NO: 20), reverse primer 5′-TGC AGG CAC TGA AGC ACC A (SEQ ID NO: 21); and mCPT2, forward primer 5′-CCA GGG CTT TGA CCG ACA CTT GT (SEQ ID NO: 22), reverse primer 5′-GCC AAA GCC ATC AGG GAC CA (SEQ ID NO: 23). The PCR primers for human CPT1A, CPT1B, CPT1C and CPT2 were as follows: hCPT1A, forward primer 5′-AGA AAT GTC GCA CGA GCC CAG AC (SEQ ID NO: 24), reverse primer 5′-CCA TGG CCC GCA CGA AGT C (SEQ ID NO: 25); hCPT1B, forward primer 5′-CTT TGG CCC TGT AGC AGA TGA (SEQ ID NO: 26), reverse primer 5′-TCG TCT CTG AGC TTG AGA ACT T (SEQ ID NO: 27); hCPT1C, forward primer 5′-CGC GCT GTT TGC CTC GTG TTT GT (SEQ ID NO: 28), reverse primer 5′-CGG CCA GAG AAG ATG CGG ACC AG (SEQ ID NO: 29); hCPT2, forward primer 5′-AAG AGA CTC ATA CGC TTT GTG C (SEQ ID NO: 30), reverse primer 5′-GGG TTT GGG TAA ACG AGT TGA (SEQ ID NO: 31); and Hif1a, forward primer 5′-CCA GAT CTC GGC GAA GTA A (SEQ ID NO: 32), reverse primer 5′-CCT CAC ACG CAA ATA GCT G (SEQ ID NO: 33).

The real time PCR was performed on an SDS 7900 (BD) with SYBR Green fluorescence (Applied Biosystems). The samples were normalized to the stably expressed reference gene GAPDH.

In situ hybridization—In situ hybridization was performed as previously described {Skinnider, B. F. et al. Interleukin 13 and interleukin 13 receptor are frequently expressed by Hodgkin and Reed-Sternberg cells of Hodgkin lymphoma. Blood 97, 250-5 (2001); Hui, C. C. & Joyner, A. L. A mouse model of greig cephalopolysyndactyly syndrome: the extra-toesJ mutation contains an intragenic deletion of the Gli3 gene. Nat Genet. 3, 241-6 (1993).}. Briefly, E14.5 embryos of C57/b16 p53+/− and p53−/− mice were sham-irradiated or subjected in utero to 5Gy X-ray irradiation. At 8 hrs post-irradiation, recovered embryos were dissected, fixed in 4% paraformaldehyde, processed and embedded in paraffin. Tissue sections (4-6 mm) were cut, deparaffinized, acetylated and exposed to ³³P-UTP-labelled riboprobes. The Cpt1c cDNA template from which the riboprobes were made was a 700 bp fragment cloned into pBluescript SK (Invitrogen). The p21 cDNA template was a full-length fragment. Sense and antisense probes were synthesized from linearized templates using T3 or T7 RNA polymerase, labeled with [a³³P]-UTP (Amersham), and processed as previously described {Skinnider, B. F. et al. Interleukin 13 and interleukin 13 receptor are frequently expressed by Hodgkin and Reed-Sternberg cells of Hodgkin lymphoma. Blood 97, 250-5 (2001); Hui, C. C. & Joyner, A. L. A mouse model of greig cephalopolysyndactyl)-syndrome: the extra-toesJ mutation contains an intragenic deletion of the Gli3 gene. Nat Genet. 3, 241-6 (1993).}.
In Vivo Hypoxia Exposure—The breast cancer mouse model MMTV-PyMT^{634Mul} was bred and maintained at the Animal Resource Centre of the Ontario Cancer Institute in compliance with the guidelines of the Canadian Council on Animal Care. Tissue samples were obtained at 2 weeks-of-age and screened for the presence of the PyMT transgene using the following primers (forward) 5′ GGA AGC AAG TAC TTC ACA AGG 3′ (SEQ ID NO: 34) and (reverse) 5′ GGA AAG TCA CTA GGA GCA GGG 3′ (SEQ ID NO: 35). Mice were weaned at 3 weeks-of-age. At 3 months-of-age, tumour-bearing females were randomly allocated to either chronic hypoxia (n=5) or air control (n=4) groups and sealed into air-tight chambers (Billups-Rothenberg Inc., Del Mar, Calif.) flushed with humidified, 7% oxygen, balance nitrogen gas mixture or air, respectively. To determine the levels of tumour hypoxia, the mice were injected intraperitoneally with 0.01 mL/g of 10 mM EF5 ([2-(2-nitro-1H-imidazol-1-yl)-N-(2,2,3,3,3-pentafluoropropyl acetamide], provided by Dr. Cameron Koch, University of Pennsylvania) prior to the gassing exposure. Following an average of 3.5 hours of gassing, the mice were killed, starting with the chronic hypoxia exposed mice, and tumours (<10 mm in diameter) were excised and snap frozen in liquid nitrogen.

The levels of EF5 were quantified immunohistochemically using the antibody ELK3-51 (1/50 O/N R/T; provided by Dr. Cameron Koch, University of Pennsylvania). Total area of positive staining in tumour sections, with areas of necrosis and connective tissue excluded, was quantified with the positive pixel algorithm by Aperio ImageScope (Aperio Technologies, Vista, Calif.). In situ analysis of Cpt1c expression in these tumors was performed as described above.

In situ analysis of Cpt1c expression in tumors was done as described above.

Cell cycle and cell death analyses—Cell cycle analysis of ES cells was performed using the BrdU Flow Kit (BD Bioscience Rockville, Md.) according to the manufacturer's protocol. Shifts in mitochondrial membrane potential were detected using a standard protocol and JC-1 (5,5′,6,6′-tetrachloro-1,1′,3,3′ tetraethylbenzimidazolylcarbocyanine iodide/chloride) (Stratagene, La Jolla, Calif.). Cell fluorescence was detected using a flow cytometer (FACSCalibur™, Becton Dickinson, San Jose), CellQuest™ and FlowJo software according to standard protocols. Active caspase-3 was detected by flow cytometry using the BD Bioscience kit according to the manufacturer's instructions (BD PharMingen, #559565). Cleaved caspase-9 was detected using carboxy-fluorescein-labeled caspase inhibitors (B-Bridge International Inc.) according to the manufacturer's instructions. Apoptosis was measured using standard protocols employing Annexin V and propidium iodide (BD Bioscience).
Sulforhodamine B (SRB) assay—A549 and MCF-7 cells were fixed in situ by gently aspirating off the culture medium and adding 50 μl cold 10% trichloroacetic acid (TCA) per well and incubating at 4° C. for 30-60 min. The plates were washed 5 times with tap water and allowed to air dry for 5 min. SRB solution (50 μl of a 0.4% w/v preparation) dissolved in 1% (v/v) acetic acid was added to each well. Plates were incubated at room temperature for 30 min, washed four times with 1% acetic acid to remove any unbound dye, and air-dried for 5 min. SRB stains were then solubilized by adding 100 μl 10 mM Tris-HCl, pH 10.5 to each well. Absorbance was read at 570 nm.
Electron microscopy—Cpt1c^{gt/gt} cells and Cpt1c^{+/gt} cells were fixed in 0.1M phosphate buffer containing 4% formaldehyde and 0.5% glutaraldehyde, and treated with 1% osmium tetraoxide. After dehydration in an ethanol gradient series followed by a polymerization step, tissue sections of 70 nm were obtained and examined using standard protocols.
Xenograft models—The pRS (retroviral-silencing)-shCPT1C gene-specific shRNA expression cassette (sense insert sequence 5′-CGGACTATGTTTCCTCAGGCGGTGGATTC-3′ (SEQ ID NO: 36)), and the control shRNA plasmid, pRS-shGFP (TR30001), were purchased from Origene (Rockland, Md.). Amphotropic Phoenix packaging cells (ATCC, Manassas, Va.) were transiently transfected with either pRS-shGFP or pRS-shCPT1C by using FuGENE 6 transfection reagent (Roche Diagnostics, Indianapolis, Ind.). Culture supernatants were collected 2 days after transfection and filtered through 0.45-μm pore-size filters. MDA-MB-468 breast cancer cells (ATCC, Manassas, Va.) were infected with retroviruses by culturing the cells for 24 hours in 1:1 Phoenix conditioned media (Dulbecco's Modified Eagle's Media, 10% FCS, supplemented with 8 μg/ml Polybrene; Sigma-Aldrich). This transfection process was repeated three times to increase the transfection efficiency. One day after the final infection, the pRS-shGFP and pRS-shCPT1C infected MDA-MB-468 cells were trypsinized, counted and injected subcutaneously into the left and right hindlimb, respectively, of nude mice at concentrations of 1.25×10⁶ and 5×10⁶ cells (5 mice per group). The tumors were measured and viable tumor area was calculated twice weekly for approximately 10 weeks.
Statistics—The paired t-test and unpaired t-test were used for comparisons where appropriate. P values were Bonferroni-corrected for multiple comparisons. P<0.05 was considered significant. Analyses were performed using StatView Version 5 (SAS Institute, Chicago, Ill.).

Hypoxia, a physiological state in which oxygen is limited, is known to be associated with the patho-physiology of many diseases. These include strokes, inflammation and autoimmune diseases, and cancers. The foregoing experimental results establish a pathway controlling hypoxia-induced cell death controlled by a mitochondrial associated enzyme carnitine palmitoyltransferase 1C. CPT1C is upregulated by hypoxia which aids cell survival under this stress, possibly by increasing fatty acid oxidation, thus energy supply. Since the absence of CPT-1C induces cell death and molecular or genetic depletion of CPT1C sensitizes cancer cells to hypoxia, inhibition of CPT1C, either chemically or genetically, will increase the susceptibility of cancer to hypoxia-induced stress, cell death and thus have anti-tumor activity.

The foregoing results also establish that the antitumor effect of reducing or inhibiting CPT1C activity in cells can be augmented by inhibiting glycolysis of the cells.

Solid tumors frequently contain regions of poor oxygenation and high acidity {Gatenby, R. A. & Gillies, R. J. Why do cancers have high aerobic glycolysis? Nat Rev Cancer 4, 891-9 (2004)}¹². The hypoxia in such tumors can act in an epigenetic fashion to induce changes in gene expression and glucose metabolism that promote tumor cell survival. Only tumor cells capable of developing an unusual tolerance to both limited oxygen availability and acidosis resulting from excessive lactate production will survive. This survival is widely believed to depend on increased glycolysis and many glycolytic enzymes are upregulated in cancers {Pelicano, H., Martin, D. S., Xu, R. H. & Huang, P. Glycolysis inhibition for anticancer treatment. Oncogene 25, 4633-46 (2006)}. However, when glucose becomes limiting due to a restricted blood supply, precursors of proteins, nucleic acids and other structural components may become alternative energy sources for cancers. Here, CPT1C has been identified as a gene that is induced by either p53 or low oxygen and that regulates hypoxia-induced cell death. Consistent with this, depletion of CPT1C in cancer cells using siRNA results in decrease in ATP production.

The results demonstrate that CPT1C is a novel p53-regulated gene that protects both human and murine cells from metabolic stress. siRNA knockdown of CPT1C in human cancer cell lines increases cell death under conditions of hypoxia or limiting glucose, and CPT1C mRNA is upregulated in human lung tumor samples. A loss-of-function gene trap mutation of Cpt1c in murine ES cells (Cpt1c^{gt/gt} cells) leads to mitochondrial swelling and mitochondrial membrane abornormality, altered lipid metabolism, decreased proliferation, and increased apoptosis due to spontaneous activation of the mitochondrial apoptotic pathway. Thus, both normal and cancerous cells depleted of CPT1C undergo spontaneous cell death under hypoxic and low glucose conditions.

CPT1C is upregulated in a p53-dependent manner in vitro as well as in vivo, perhaps due to the function of a conserved enhancer element in the first intron of the Cpt1c gene that binds to p53 directly and regulates Cpt1c transcription. However, in contrast to other p53-regulated genes encoding mitochondrial proteins, loss of CPT1C function decreases mitochondrial membrane potential and spontaneously induces the mitochondrial apoptosis pathway by activating caspase-3 and -9. In addition, the mitochondria of Cpt1c^{gt/gt} cells are enlarged and show disruption of internal structure. Even under normal culture conditions, Cpt1c^{gt/gt} cells show both mitochondrial swelling and a cytoplasmic accumulation of lipid droplets that is not present in Cpt1c^{+/gt} cells. Fatty acid analyses show that the Cpt1c-deficient ES cells exhibit altered lipid metabolism evidenced by the altered abundance of several major fatty acids as well as phosphoglycerides. These observations suggest either that CPT1C is involved in the metabolism of lipids that are important for the stability and functionality of the mitochondrial membranes, or that these lipid droplets are toxic to the mitochondria. Indeed, the accumulation of certain long-chain FA in a cell promotes its apoptosis {Feldkamp, T., Kribben, A., Roeser, N. F., Senter, R. A. & Weinberg, J. M. Accumulation of nonesterified fatty acids causes the sustained energetic deficit in kidney proximal tubules after hypoxia-reoxygenation. Am J Physiol Renal Physiol 290, F465-77 (2006)}. In addition, Cpt1c^{gt/gt} cells exhibit slower growth and smaller size than Cpt1c^{+/gt} cells. Surprisingly, the treatment of Cpt1c^{gt/gt} and Cpt1c^{+/gt} cells with stimuli known to activate p53 did not reveal any additional differences with respect to cell death. However, Cpt1c^{gt/gt} cells are much more sensitive to hypoxia than are Cpt1c^{+/gt} cells. Previous reports did not identify CPT1C as a p53 target gene induced by hypoxia but we have observed that there are only very low levels of CPT1C mRNA in the cell types examined in these studies (data not shown). The results imply that CPT1C may act as a direct functional link between p53 and responses to hypoxia.

Here, it has been determined that CPT1C is a bona fide p53 target gene that promotes cell survival, particularly under conditions of metabolic stress. Real time RT-PCR analyses in multiple cell lines show that, of all CPT1 family members, only expression of the CPT1C isoform is p53-dependent. Furthermore, in response to p53 activation, CPT1C is expressed in all murine adult tissues and in ES cells. Interestingly, hypoxia-induction of CPT1C expression in human cancer cells does not seem to be entirely dependent on p53 as evidenced by its upregulation observed in HCT116 p53 null cells as well as in human lung tumors that are negative for p53 expression. These findings suggest that, as well as in its expression pattern, CPT1C may be unique among CPT1 family members in its function. It has been previously hypothesized that CPT1C might utilize substrates distinct from those of other CPT1 enzymes, since CPT1C ectopically expressed in yeast showed no catalytic activity against common acyl esters (unlike other CPT1 family members) {Price, N. et al. A novel brain-expressed protein related to carnitine palmitoyltransferase I. Genomics 80, 433-42 (2002)}. However, the FAO experiment demonstrates that CPT1C may be able to use long chain fatty acids as substrates, at least in the cancer cells. This finding and identification of fatty acids altered in the Cpt1c-deficient ES cells should facilitate elucidation of physiological substrates of CPT1C.

In addition to the striking changes in glucose metabolism that occur in solid tumors, studies of human cancer patients suggest that there is often an increase in free FA turnover, oxidation and clearance in these malignancies {Russell, S. T. & Tisdale, M. J. Effect of a tumour-derived lipid-mobilising factor on glucose and lipid metabolism in vivo. Br J Cancer 87, 580-4 (2002)}. Fatty acids are synthesized de novo by fatty acid synthase (FAS), and very long chain FA generated by FAS are required for cell division {Hannun, Y. A. & Obeid, L. M. The Ceramide-centric universe of lipid-mediated cell regulation: stress encounters of the lipid kind. J Biol Chem 277, 25847-50 (2002)}. Importantly, tumors overexpressing FAS are more aggressive than tumors with normal FAS levels {Rossi, S. et al. Fatty acid synthase expression defines distinct molecular signatures in prostate cancer. Mol Cancer Res 1, 707-15 (2003)}, indicating that FAS overexpression can confer a selective growth advantage. Here, Cpt1c^{gt/gt} cells showed enhanced sensitivity to hypoxia, a phenotype that was gene dosage-dependent under conditions of low glucose. These data thus contribute to the growing evidence that FAO may drive tumor expansion in a hypoxic environment, perhaps by enabling cells to use fatty acids as fuel source.

Tumor cells that become hypoxic can develop resistance to selective therapies. A treatment that counters this tendency and increases the sensitivity of hypoxic tumor cells to drug treatment might therefore be a useful therapeutic strategy. Results disclosed herein suggested that CPT1C is a gene that confers survival during hypoxia, and that decreased CPT1C activity can sensitize cancer cells to hypoxia-induced death. The results show that CPT1C is induced by hypoxia and that depletion of CPT1C in cancer cells reduces their viability, especially under conditions of metabolic stress such as hypoxia or glucose deprivation. It has also been demonstrated that CPT1C expression is substantially upregulated in human lung tumors, supporting the notion that CPT1C contributes to cancer cell survival in vivo. CPT1C depletion was also shown to substantially suppress tumor growth in xenograft models. Taken together, these findings suggest that CPT1C may be an attractive target for therapeutic intervention for tumours that are hypoxic and deprived of carbohydrate sources of nutrient.

A particular embodiment of the invention disclosed herein includes nucleic acid therapeutic agents and methods for inhibiting or reducing gene expression of CPT1C. By “inhibit,” “reduce,” or “downregulate,” it is meant that the expression of the CPT1C gene, or level of RNAs or the equivalent RNA-encoded protein, or activity of such encoded protein (such as CPT1C protein), is reduced below the corresponding level observed in the absence of the nucleic acid molecules of the invention. In certain embodiments, inhibition or down-regulation of CPT1C with siRNA molecules is below that level observed in the presence of, for example, an oligonucleotide with a random sequence or with mismatches.

The present invention includes therapies involving methods for inhibiting or reducing gene expression of CPT1C in combination with other therapeutic approaches, specifically those which operate through a differently mediated cellular apoptotic/survival or proliferation regulatory pathway, and in particular those therapeutic strategies that inhibit glucose utilization in glycolysis. See, for example, Pelicano H, et al., “Glycolysis Inhibition for anticancer treatment,” Oncogene. 2006 (25): 4633-4646; Hatzivassiliou G, et al., Cancer Cell. 2005 October; 8(4):311-21, Liu Y., “Fatty acid oxidation is a dominant bioenergetic pathway in protstate cancer,” Prostate Cancer Prostatic Des. 2006 May 9 [published electronically], WO 2006/020403, WO 2006/017494, and WO 2004/100885. Another combination would be one which includes mTOR inhibition that attentuates glucose metabolism and induces apoptosis. Such treatments would include derivatives of the known mTOR inhibitor, rapamycin. See Majumder et al, Nature Medicine, 2004 May 23; 10(6):594, WO 2006/050461, WO 2004/004644 and WO 2003/053223.

With respect to such combination therapies, particular glycolysis inhibitors of the invention include, but are not limited to 2-deoxyglucose, lonidamine, 3-bromopyruvate, imatinib and oxythiamine. Of these, 3-bromopyruvate which is relatively selective for hexokinase of the primary phase of the glycolytic pathway, is especially contemplated.

The present invention employs compounds, preferably oligonucleotides and similar species for use in modulating the function or effect of nucleic acid molecules encoding CPT1C. This is accomplished by providing oligonucleotides which specifically hybridize with one or more nucleic acid molecules encoding CPT1C, specifically mRNA encoding CPT1C, i.e. having the nucleotide sequence identified as SEQ ID NO:1 shown in FIG. 1(E). Thus “target nucleic acid” refers to a nucleic acid molecule encoding CPT1C. As used herein, the term “nucleic acid molecule encoding CPT1C” has been used for convenience to encompass DNA encoding CPT1C, RNA (including pre-mRNA and mRNA or portions thereof) transcribed from such DNA, and also cDNA derived from such RNA. The hybridization of a compound of this invention with its target nucleic acid is generally referred to as “antisense”. Consequently, the preferred mechanism believed to be included in the practice of some preferred embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

As used herein, the term “nucleic acid therapeutic agent” or “nucleic acid agent” or “nucleic acid compound” refers to any nucleic acid-based compound that contains nucleotides and has a desired effect on the target nucleic acid molecule. The nucleic acid therapeutic agents can be single-, double-, or multiple-stranded, and can comprise modified or unmodified nucleotides or non-nucleotides or various mixtures, and combinations thereof. Examples include an antisense molecule, an RNAi construct (e.g., an siRNA molecule), or a ribozyme. In certain specific embodiments, nucleic acid therapeutic agents of the disclosure are directed to siRNA nucleic acid compounds against CPT1C.

The present invention thus includes the use of siRNAs to reduce the amount of cellular CPT1C. Recent studies have suggested that siRNAs may be used as drugs for the silencing of a gene in certain cases. The idea behind this is similar to that of antisense molecules as therapeutic agents. The mechanism of action of antisense RNA and the current state of the art on use of antisense tools is reviewed in Kumar et al (1998): Antisense RNA: function and fate of duplex RNA in cells of higher eukaryotes. Microbiol. Mol Biol Rev. 1998 December; 62(4):1415-34. There are reviews on the chemical aspects (Crooke, 1995: Progress in antisense therapeutics. Hematol Pathol. 1995; 9(2):59-72.; Uhlmann et al, 1990), cellular aspects (Wagner, 1994: Gene inhibition using antisense oligodeoxynucleotides. Nature. 1994 Nov. 24; 372(6504):333-5.) and therapeutic aspects (Hanania, et al, 1995: Recent advances in the application of gene therapy to human disease. Am J. Med. 1995 November; 99(5):537-52; Scanlon, et al, 1995: Oligonucleotide-mediated modulation of mammalian gene expression. FASEB J. 1995 October; 9(13):1288-96; Gewirtz, 1993: Oligodeoxynucleotide-based therapeutics for human leukemias. Stem Cells. 1993 October; 11 Suppl 3:96-103) of this rapidly developing technology. The use of antisense oligonucleotides in inhibition of various genes has been described in Yeh et al (1998): Inhibition of BMP receptor synthesis by antisense oligonucleotides attenuates OP-1 action in primary cultures of fetal rat calvaria cells. J Bone Miller Res. 1998 December; 13(12):1870-9; Meiri et al (1998) Memory and long-term potentiation (LTP) dissociated: normal spatial memory despite CA1 LTP elimination with Kv1.4 antisense. Proc Natl Acad Sci USA. 1998 Dec. 8; 95(25):15037-42; Kondo et al (1998): Antisense telomerase treatment: induction of two distinct pathways, apoptosis and differentiation. FASEB J. 1998 July; 12(10):801-11; Stix (1998): Shutting down a gene. Antisense drug wins approval. Sci Am. 1998 November; 279(5):46, 50; Flanagan (1998) Antisense comes of age. Cancer Metastasis Rev. 1998 June; 17(2):169-76; Guinot et al (1998) Antisense oligonucleotides: a new therapeutic approach Pathol Biol (Paris). 1998 May; 46(5):347-54, and references therein. The methods described therein also apply generally to delivery of siRNAs. A recent review of the use of siRNAs in cancer treatment is given by Putral et al. in Drug News Perspect. 2006 July-August; 19(6):317-24.

Recently, delivery systems aimed specifically at the enhanced and improved delivery of siRNA into mammalian cells have been developed. Shen et al. (FEBS letters 539: 111-114 (2003)) described an adenovirus-based vector which efficiently delivers siRNAs into mammalian cells. Additional detail on viral-based siRNA delivery systems can be found in Xia et al., Nature Biotechnology 20: 1006-1010 (2002); and Reich et al., Molecular Vision 9: 210-216 (2003).

Sorensen et al. (J. Mol. Biol. 327: 761-766 (2003)) devised injection-based systems for systemic delivery of siRNAs to adult mice, by cationic liposome-based intravenous injection and/or intraperitoneal injection.

A system for efficient delivery of siRNA into mice by rapid tail vain injection has also been developed (Lewis et al., Nature Genetics 32: 107-108 (2002)).

Additionally, the peptide based gene delivery system MPG, previously used for DNA targeting, has been modified to be effective with siRNAs (Simeoni et al., Nuclaic Acids Research 31, 11: 2717-2724 (2003)).

Any method for gene inactivation may be used with existing or later derived methods which can be adapted to work as part of the present invention.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. One preferred result of such interference with target nucleic acid function is modulation of the expression of CPT1C. In the context of the present invention, “modulation” and “modulation of expression” mean either an increase (stimulation) or a decrease (inhibition) in the amount or levels of a nucleic acid molecule encoding the gene, e.g., DNA or RNA. Inhibition is often the preferred form of modulation of expression and mRNA is often a preferred target nucleic acid.

In the context of this invention, “hybridization” means the pairing of complementary strands of oligomeric compounds. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

In the present invention the phrase “stringent hybridization conditions” or “stringent conditions” refers to conditions under which a compound of the invention will hybridize to its target sequence, but to a minimal number of other sequences. Stringent conditions are sequence-dependent and will be different in different circumstances and in the context of this invention, “stringent conditions” under which oligomeric compounds hybridize to a target sequence are determined by the nature and composition of the oligomeric compounds and the assays in which they are being investigated.

“Complementary,” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

It is understood in the art that the sequence of an antisense compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure or hairpin structure). It is preferred that the antisense compounds of the present invention comprise at least 70% sequence complementarity to a target region within the target nucleic acid, more preferably that they comprise 90% sequence complementarity and even more preferably comprise 95% sequence complementarity to the target region within the target nucleic acid sequence to which they are targeted. For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

According to the present invention, compounds include antisense oligomeric compounds, antisense oligonucleotides, ribozymes, external guide sequence (EGS) oligonucleotides, alternate splicers, primers, probes, and other oligomeric compounds which hybridize to at least a portion of the target nucleic acid. As such, these compounds may be introduced in the form of single-stranded, double-stranded, circular or hairpin oligomeric compounds and may contain structural elements such as internal or terminal bulges or loops. Once introduced to a system, the compounds of the invention may elicit the action of one or more enzymes or structural proteins to effect modification of the target nucleic acid.

One non-limiting example of such an enzyme is RNAse H, a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While the preferred form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals and is believed to have an evolutionary connection to viral defense and transposon silencing.

The first evidence that dsRNA could lead to gene silencing in animals came in 1995 from work in the nematode, Caenorhabditis elegans (Guo and Kempheus, Cell, 1995, 81, 611-620). Montgomery et al. have shown that the primary interference effects of dsRNA are posttranscriptional (Montgomery et al., Proc. Natl. Acad. Sci. USA, 1998, 95, 15502-15507). The posttranscriptional antisense mechanism defined in Caenorhabditis elegans resulting from exposure to double-stranded RNA (dsRNA) has since been designated RNA interference (RNAi). This term has been generalized to mean antisense-mediated gene silencing involving the introduction of dsRNA leading to the sequence-specific reduction of endogenous targeted mRNA levels (Fire et al., Nature, 1998, 391, 806-811). Recently, it has been shown that it is, in fact, the single-stranded RNA oligomers of antisense polarity of the dsRNAs which are the potent inducers of RNAi (Tijsterman et al., Science, 2002, 295, 694-697).

Certain embodiments of the invention thus related to double-stranded RNA (dsRNA). The term “dsRNA” as used herein refers to a double-stranded RNA molecule capable of RNA interference (RNAi), including siRNA. See for example, Bass, 2001, Nature, 4 11, 428-429; Elbashir et al., 2001, Nature, 4 11, 494-498; and Kreutzer et al., PCT Publication No. WO 00/44895; Zernicka-Goetz et al., PCT Publication No. WO 01/36646; Fire, PCT Publication No. WO 99/3261 9; Plaetinck et al., PCT Publication No. WO 00/01846; Mello and Fire, PCT Publication No. WO 01/29058; Deschamps-Depaillette, PCT Publication No. WO 99/07409; and Li et al., PCT Publication No. WO 00/44914. RNAi is a term initially applied to a phenomenon observed in plants and worms where a dsRNA blocks gene expression in a specific and post-transcriptional manner. RNAi provides a useful method of inhibiting gene expression in vitro or in vivo.

The oligonucleotides of the present invention also include variants in which a different base is present at one or more of the nucleotide positions in the oligonucleotide. For example, if the first nucleotide is an adenosine, variants may be produced which contain thymidine, guanosine or cytidine at this position. This may be done at any of the positions of the oligonucleotide. These oligonucleotides are then tested using the methods described herein to determine their ability to inhibit expression of CPT1C.

In the context of this invention, the term “oligomeric compound” refers to a polymer or oligomer comprising a plurality of monomeric units. In the context of this invention, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

While oligonucleotides are a preferred form of the compounds of this invention, the present invention comprehends other families of compounds as well, including but not limited to oligonucleotide analogs and mimetics such as those described herein.

The compounds in accordance with this invention preferably comprise from about 8 to about 80 nucleobases (i.e. from about 8 to about 80 linked nucleosides). One of ordinary skill in the art will appreciate that the invention embodies compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

In one preferred embodiment, the compounds of the invention are 12 to 50 nucleobases in length. One having ordinary skill in the art will appreciate that this embodies compounds of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases in length.

In another preferred embodiment, the compounds of the invention are 15 to 30 nucleobases in length. One having ordinary skill in the art will appreciate that this embodies compounds of 15, 16, 17, 18, 19, 20, 21, 22, 23, 24, 25, 26, 27, 28, 29, or 30 nucleobases in length.

Particularly preferred compounds are oligonucleotides from about 12 to about 50 nucleobases, even more preferably those comprising from about 15 to about 30 nucleobases.

Antisense compounds 8-80 nucleobases in length comprising a stretch of at least eight (8) consecutive nucleobases selected from within the illustrative antisense compounds are considered to be suitable antisense compounds as well.

Exemplary preferred antisense compounds include oligonucleotide sequences that comprise at least the 8 consecutive nucleobases from the 5′-terminus of one of the illustrative preferred antisense compounds (the remaining nucleobases being a consecutive stretch of the same oligonucleotide beginning immediately upstream of the 5′-terminus of the antisense compound which is specifically hybridizable to the target nucleic acid and continuing until the oligonucleotide contains about 8 to about 80 nucleobases). Similarly preferred antisense compounds are represented by oligonucleotide sequences that comprise at least the 8 consecutive nucleobases from the 3′-terminus of one of the illustrative preferred antisense compounds (the remaining nucleobases being a consecutive stretch of the same oligonucleotide beginning immediately downstream of the 3′-terminus of the antisense compound which is specifically hybridizable to the target nucleic acid and continuing until the oligonucleotide contains about 8 to about 80 nucleobases). One having skill in the art armed with the preferred antisense compounds illustrated herein will be able, without undue experimentation, to identify further preferred antisense compounds.

“Targeting” an antisense compound to a particular nucleic acid molecule, in the context of this invention, can be a multistep process. The process usually begins with the identification of a target nucleic acid whose function is to be modulated. This target nucleic acid may be, for example, a cellular gene (or mRNA transcribed from the gene) whose expression is associated with a particular disorder or disease state, or a nucleic acid molecule from an infectious agent. In the present invention, the target nucleic acid encodes CPT1C.

The targeting process usually also includes determination of at least one target region, segment, or site within the target nucleic acid for the antisense interaction to occur such that the desired effect, e.g., modulation of expression, will result. Within the context of the present invention, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as positions within a target nucleic acid.

Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA transcribed from a gene encoding CPT1C, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively).

The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the antisense compounds of the present invention.

The open reading frame (ORF) or “coding region,” which is known in the art to refer to the region between the translation initiation codon and the translation termination codon, is also a region which may be targeted effectively. Within the context of the present invention, a preferred region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also preferred to target the 5′ cap region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. Targeting splice sites, i.e., intron-exon junctions or exon-intron junctions, may also be particularly useful in situations where aberrant splicing is implicated in disease, or where an overproduction of a particular splice product is implicated in disease. Aberrant fusion junctions due to rearrangements or deletions are also preferred target sites. mRNA transcripts produced via the process of splicing of two (or more) mRNAs from different gene sources are known as “fusion transcripts”. It is also known that introns can be effectively targeted using antisense compounds targeted to, for example, DNA or pre-mRNA.

It is also known in the art that alternative RNA transcripts can be produced from the same genomic region of DNA. These alternative transcripts are generally known as “variants”. More specifically, “pre-mRNA variants” are transcripts produced from the same genomic DNA that differ from other transcripts produced from the same genomic DNA in either their start or stop position and contain both intronic and exonic sequence.

Upon excision of one or more exon or intron regions, or portions thereof during splicing, pre-mRNA variants produce smaller “mRNA variants”. Consequently, mRNA variants are processed pre-mRNA variants and each unique pre-mRNA variant must always produce a unique mRNA variant as a result of splicing. These mRNA variants are also known as “alternative splice variants”. If no splicing of the pre-mRNA variant occurs then the pre-mRNA variant is identical to the mRNA variant.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Within the context of the invention, the types of variants described herein are also preferred target nucleic acids.

The locations on the target nucleic acid to which the preferred antisense compounds hybridize are hereinbelow referred to as “preferred target segments.” As used herein the term “preferred target segment” is defined as at least an 8-nucleobase portion of a target region to which an active antisense compound is targeted. While not wishing to be bound by theory, it is presently believed that these target segments represent portions of the target nucleic acid which are accessible for hybridization.

While the specific sequences of certain preferred target segments are set forth herein, one of skill in the art will recognize that these serve to illustrate and describe particular embodiments within the scope of the present invention. Additional preferred target segments may be identified by one having ordinary skill. Design of siRNAs based on the mRNA sequence can be accomplished using commercial products designed therefor as available from, for example, Ambion of Applied Biosystems, headquartered in Foster City, Calif., U.S.A. (ambion.com). The siRNA can be a single-stranded hairpin polynucleotide having self-complementary sense and antisense regions, wherein the antisense region comprises complementarity to a target nucleic acid compound. The siRNA can be a circular single-stranded polynucleotide having two. or more loop structures and a stem comprising self-complementary sense and antisense regions, wherein the antisense region comprises complementarity to a target nucleic acid, and wherein the circular polynucleotide can be processed either in vivo or in vitro to generate an active siRNA capable of mediating RNAi. The siRNA can also comprise a single-stranded polynucleotide having complementarity to a target nucleic acid, wherein the single-stranded polynucleotide can further comprise a terminal phosphate group, such as a 5′-phosphate (see for example Martinez et al., 2002, Cell., 110, 563-574), or 5′,3′-diphosphate.

Target segments 8-80 nucleobases in length comprising a stretch of at least eight (8) consecutive nucleobases selected from within the illustrative preferred target segments are considered to be suitable for targeting as well.

Target segments can include DNA or RNA sequences that comprise at least the 8 consecutive nucleobases from the 5′-terminus of one of the illustrative preferred target segments (the remaining nucleobases being a consecutive stretch of the same DNA or RNA beginning immediately upstream of the 5′-terminus of the target segment and continuing until the DNA or RNA contains about 8 to about 80 nucleobases). Similarly preferred target segments are represented by DNA or RNA sequences that comprise at least the 8 consecutive nucleobases from the 3′-terminus of one of the illustrative preferred target segments (the remaining nucleobases being a consecutive stretch of the same DNA or RNA beginning immediately downstream of the 3′-terminus of the target segment and continuing until the DNA or RNA contains about 8 to about 80 nucleobases). One having skill in the art armed with the preferred target segments illustrated herein will be able, without undue experimentation, to identify further preferred target segments.

Once one or more target regions, segments or sites have been identified, antisense compounds are chosen which are sufficiently complementary to the target, i.e., hybridize sufficiently well and with sufficient specificity, to give the desired effect.

In a further embodiment, the “preferred target segments” identified herein may be employed in a screen for additional compounds that modulate the expression of CPT1C. “Modulators” are those compounds that decrease or increase the expression of a nucleic acid molecule encoding CPT1C and which comprise at least an 8-nucleobase portion which is complementary to a preferred target segment. The screening method comprises the steps of contacting a preferred target segment of a nucleic acid molecule encoding CPT1C with one or more candidate modulators, and selecting for one or more candidate modulators which decrease or increase the expression of a nucleic acid molecule encoding CPT1C. Once it is shown that the candidate modulator or modulators are capable of modulating (e.g. either decreasing or increasing) the expression of a nucleic acid molecule encoding CPT1C, the modulator may then be employed in further investigative studies of the function of CPT1C, or for use as a research, diagnostic, or therapeutic agent in accordance with the present invention.

The preferred target segments of the present invention may be also be combined with their respective complementary antisense compounds of the present invention to form stabilized double-stranded (duplexed) oligonucleotides.

Such double stranded oligonucleotide moieties have been shown in the art to modulate target expression and regulate translation as well as RNA processing via an antisense mechanism. Moreover, the double-stranded moieties may be subject to chemical modifications (Fire et al., Nature, 1998, 391, 806-811; Timmons and Fire, Nature 1998, 395, 854; Timmons et al., Gene, 2001, 263, 103-112; Tabara et al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200). For example, such double-stranded moieties have been shown to inhibit the target by the classical hybridization of antisense strand of the duplex to the target, thereby triggering enzymatic degradation of the target (Tijsterman et al., Science, 2002, 295, 694-697).

In a further specific embodiment, the siRNA is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

PCT application WO 01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for a dsRNA of interest, wherein the two overlapping transcription units yield both sense and antisense RNA transcripts from the same transgene fragment in a host cell.

Examples of the subject siRNA compounds are shown in Table IV below.

TABLE IV
Examples of siRNA molecules against CPT1C

| | |
|---|---|
| SEQ ID NO: 8 | 5′ GAA AUC CGC UGA UGG UGA A 3′ (sense) |
| | 3′ CUU UAG GCG ACU ACC ACU U 5′ (antisense) |
| SEQ ID NO: 9 | 5′ GAC AAA UCC UUC ACC CUA A 3′ (sense) |
| | 3′ CUG UUU AGG AAG UGG GAU U 5′ (antisense) |
| SEQ ID NO: 10 | 5′ AAA GGC AUC UCU CAC GUU U 3′ (sense) |
| | 3′ UUU CCG UAG AGA GUG CAA A 5′ (antisense) |
| SEQ ID NO: 11 | 5′ GAG GGA GGC CUG CAA CUU U 3′ (sense) |
| | 3′ CUC CCU CCG GAC GUU GAA A 5′ (antisense) |

The compounds of the present invention can also be applied in the areas of drug discovery and target validation. The present invention comprehends the use of the compounds and preferred target segments identified herein in drug discovery efforts to elucidate relationships that exist between CPT1C and a disease state, phenotype, or condition. These methods include detecting or modulating CPT1C comprising contacting a sample, tissue, cell, or organism with the compounds of the present invention, measuring the nucleic acid or protein level of CPT1C and/or a related phenotypic or chemical endpoint at some time after treatment, and optionally comparing the measured value to a non-treated sample or sample treated with a further compound of the invention. These methods can also be performed in parallel or in combination with other experiments to determine the function of unknown genes for the process of target validation or to determine the validity of a particular gene product as a target for treatment or prevention of a particular disease, condition, or phenotype.

The compounds of the present invention can be utilized for diagnostics, therapeutics, prophylaxis and as research reagents and kits. Furthermore, antisense oligonucleotides, which are able to inhibit gene expression with exquisite specificity, are often used by those of ordinary skill to elucidate the function of particular genes or to distinguish between functions of various members of a biological pathway.

For use in kits and diagnostics, the compounds of the present invention, either alone or in combination with other compounds or therapeutics, can be used as tools in differential and/or combinatorial analyses to elucidate expression patterns of a portion or the entire complement of genes expressed within cells and tissues.

As one nonlimiting example, expression patterns within cells or tissues treated with one or more antisense compounds are compared to control cells or tissues not treated with antisense compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (To, Comb. Chem. High Throughput Screen, 2000, 3, 235-41).

The compounds of the invention are useful for research and diagnostics, because these compounds hybridize to nucleic acids encoding CPT1C. For example, oligonucleotides that are shown to hybridize with such efficiency and under such conditions as disclosed herein as to be effective CPT1C inhibitors will also be effective primers or probes under conditions favoring gene amplification or detection, respectively. These primers and probes are useful in methods requiring the specific detection of nucleic acid molecules encoding CPT1C and in the amplification of said nucleic acid molecules for detection or for use in further studies of CPT1C. Hybridization of the antisense oligonucleotides, particularly the primers and probes, of the invention with a nucleic acid encoding CPT1C can be detected by means known in the art. Such means may include conjugation of an enzyme to the oligonucleotide, radiolabelling of the oligonucleotide or any other suitable detection means. Kits using such detection means for detecting the level of CPT1C in a sample may also be prepared.

The specificity and sensitivity of antisense is also harnessed by those of skill in the art for therapeutic uses. Antisense compounds have been employed as therapeutic moieties in the treatment of disease states in animals, including humans. Antisense oligonucleotide drugs, including ribozymes, have been safely and effectively administered to humans and numerous clinical trials are presently underway. It is thus established that antisense compounds can be useful therapeutic modalities that can be configured to be useful in treatment regimes for the treatment of cells, tissues and animals, especially humans.

For therapeutics, an animal, preferably a human, suspected of having a disease or disorder which can be treated by modulating the expression of CPT1C is treated by administering one or more antisense, siRNA or small molecule compounds in accordance with this invention. For example, in one non-limiting embodiment, the methods comprise the step of administering to the animal in need of treatment, a therapeutically effective amount of a CPT1C inhibitor. The CPT1C inhibitors of the present invention effectively inhibit the activity of the CPT1C target protein or inhibit the expression of the CPT1C protein. In one embodiment, the activity or expression of CPT1C in a target cell is inhibited by about 10%. Preferably, the activity or expression of CPT1C in a target cell is inhibited by about 30%. More preferably, the activity or expression of CPT1C in a target cell is inhibited by 50% or more.

For example, the reduction of the expression of CPT1C may be measured in serum, adipose tissue, liver or any other body fluid, tissue or organ of the animal. Preferably, the cells contained within said fluids, tissues or organs being analyzed contain a nucleic acid molecule encoding CPT1C protein and/or the CPT1C protein itself.

The compounds of the invention can be utilized in pharmaceutical compositions by adding an effective amount of a compound to a suitable pharmaceutically acceptable diluent or carrier. Use of the compounds and methods of the invention may also be useful prophylactically.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH.sub.2 component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference.

In other preferred oligonucleotide mimetics, both the sugar and the internucleoside linkage (i.e. the backbone), of the nucleotide units are replaced with novel groups. The nucleobase units are maintained for hybridization with an appropriate target nucleic acid. One such compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH_{Z}—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O, S- or N-alkynyl; or O-alkyl-O-alkyl, wherein the alkyl, alkenyl and alkynyl may be substituted or unsubstituted C₁ to C₁₀ alkyl or C₂ to 10 alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, Conn., CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy(2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy(also known in the art as 2′-O-dimethyl-amino-ethoxy-ethyl or 2′-DMAEOE), i.e., 2′-O—CH₂—O—CH₂—N(CH₃)₂, also described in examples hereinbelow.

Other preferred modifications include 2′-methoxy(2′-O—CH₃), 2′-aminopropoxy (2′-OCH₂CH₂CH₂NH₂), 2′-allyl(2′-CH₂—CH═CH₂), 2′-O-allyl(2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

A further preferred modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is preferably a methylene (—CH₂—)ₙ group bridging the 2′ oxygen atom and the 4′ carbon atom wherein n is 1 or 2. LNAs and preparation thereof are described in WO 98/39352 and WO 99/14226.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl(—C.ident.C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,681,941; and 5,750,692 also herein incorporated by reference.

Another modification of the oligonucleotides of the invention involves chemically linking to the oligonucleotide one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application PCT/US92/09196, filed Oct. 23, 1992, and U.S. Pat. No. 6,287,860, the entire disclosure of which are incorporated herein by reference. Conjugate moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodo-benzoic acid, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

It is not necessary for all positions in a given compound to be uniformly modified, and in fact more than one of the aforementioned modifications may be incorporated in a single compound or even at a single nucleoside within an oligonucleotide.

The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, increased stability and/or increased binding affinity for the target nucleic acid. An additional region of the oligonucleotide may serve as a substrate for enzymes capable of cleaving RNA:DNA or RNA:RNA hybrids. By way of example, RNAse H is a cellular endonuclease which cleaves the RNA strand of an RNA:DNA duplex. Activation of RNase H, therefore, results in cleavage of the RNA target, thereby greatly enhancing the efficiency of oligonucleotide-mediated inhibition of gene expression. The cleavage of RNA:RNA hybrids can, in like fashion, be accomplished through the actions of endoribonucleases, such as RNAseL which cleaves both cellular and viral RNA. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The compounds of the invention may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts, esters, or salts of such esters, or any other compound which, upon administration to an animal, including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents. The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl)phosphate] derivatives according to the methods disclosed in WO 93/24510 to Gosselin et al., published Dec. 9, 1993 or in WO 94/26764 and U.S. Pat. No. 5,770,713 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto. For oligonucleotides, preferred examples of pharmaceutically acceptable salts and their uses are further described in U.S. Pat. No. 6,287,860, which is incorporated herein in its entirety.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions of the present invention include, but are not limited to, solutions, emulsions, foams and liposome-containing formulations. The pharmaceutical compositions and formulations of the present invention may comprise one or more penetration enhancers, carriers, excipients or other active or inactive ingredients.

Emulsions are typically heterogenous systems of one liquid dispersed in another in the form of droplets usually exceeding 0.1 .mu.m in diameter. Emulsions may contain additional components in addition to the dispersed phases, and the active drug which may be present as a solution in either the aqueous phase, oily phase or itself as a separate phase. Microemulsions are included as an embodiment of the present invention. Emulsions and their uses are well known in the art and are further described in U.S. Pat. No. 6,287,860, which is incorporated herein in its entirety.

Formulations of the present invention include liposomal formulations. As used in the present invention, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein in its entirety.

The pharmaceutical formulations and compositions of the present invention may also include surfactants. The use of surfactants in drug products, formulations and in emulsions is well known in the art. Surfactants and their uses are further described in U.S. Pat. No. 6,287,860, which is incorporated herein in its entirety.

In one embodiment, the present invention employs various penetration enhancers to effect the efficient delivery of nucleic acids, particularly oligonucleotides. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein in its entirety.

One of skill in the art will recognize that formulations are routinely designed according to their intended use, i.e. route of administration.

Preferred formulations for topical administration include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

For topical or other administration, oligonucleotides of the invention may be encapsulated within liposomes or may form complexes thereto, in particular to cationic liposomes. Alternatively, oligonucleotides may be complexed to lipids, in particular to cationic lipids. Preferred fatty acids and esters, pharmaceutically acceptable salts thereof, and their uses are further described in U.S. Pat. No. 6,287,860, which is incorporated herein in its entirety. Topical formulations are described in detail in U.S. patent application Ser. No. 09/315,298 filed on May 20, 1999, which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers surfactants and chelators. Preferred surfactants include fatty acids and/or esters or salts thereof, bile acids and/or salts thereof. Preferred bile acids/salts and fatty acids and their uses are further described in U.S. Pat. No. 6,287,860, which is incorporated herein in its entirety. Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligonucleotides of the invention may be delivered orally, in granular form including sprayed dried particles, or complexed to form micro or nanoparticles. Oligonucleotide complexing agents and their uses are further described in U.S. Pat. No. 6,287,860, which is incorporated herein in its entirety. Oral formulations for oligonucleotides and their preparation are in detail in U.S. Pat. No. 6,888,906, incorporated herein by reference in its entirety.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Certain embodiments of the invention provide pharmaceutical compositions containing one or more oligomeric compounds and one or more other chemotherapeutic agents which function by a non-antisense mechanism. Examples of such chemotherapeutic agents include but are not limited to cancer chemotherapeutic drugs such as daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Combinations of antisense compounds and other non-antisense drugs are also within the scope of this invention. Two or more combined compounds may be used together or sequentially.

In another related embodiment, compositions of the invention may contain one or more antisense compounds, particularly oligonucleotides, targeted to a first nucleic acid and one or more additional antisense compounds targeted to a second nucleic acid target. Alternatively, compositions of the invention may contain two or more antisense compounds targeted to different regions of the same nucleic acid target. Numerous examples of antisense compounds are known in the art. Two or more combined compounds may be used together or sequentially.

The formulation of therapeutic compositions and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 ug to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

The contents of each document referred to in this specification are incorporated herein by reference as though reproduced herein in its entirety, and the applicant reserves the right to incorporate directly into this specification all or part of any such document. This includes the specifications of the United States provisional patent applications from which this application claims priority.

1. An isolated siRNA compound comprising at least a portion that hybridizes to a CPT1C, transcript under physiological conditions and decreases the expression of CPT1C in a cell.

2. The siRNA compound of claim 1, wherein the CPT1C transcript has a nucleotide sequence set forth in SEQ ID NO: 1.

3-14. (canceled)

15. A double stranded siRNA molecule that decreases expression of CPT1C gene, wherein each strand of said siRNA molecule is about 18 to about 30 nucleotides in length, and wherein one strand of said siRNA molecule comprises a nucleotide sequence having sufficient complementarity to an RNA of said CPT1C gene for the CPT1C molecule to direct cleavage of said RNA via RNA interference (RNAi).

16. The siRNA molecule of claim 15, wherein each strand comprises at least about 14 to 24 nucleotides that are complementary to the nucleotides of the other strand.

17-21. (canceled)

22. A pharmaceutical composition comprising an siRNA molecule of claim 16 and a pharmaceutically acceptable carrier, preferably where the composition is for use as an anticancer agent, more preferably for use in the treatment of a solid tumor, particularly lung tumor, brain tumor, prostate tumor, breast tumor, or colon tumor.

23. A method of decreasing CPT1C expression in a cell, comprising contacting the cell with an effective amount of an siRNA compound, wherein the siRNA compound comprises at least a portion that hybridizes to a CPT1C transcript under physiological conditions and decreases the expression of CPT1C in a cell.

24-26. (canceled)

27. A method for treating a tumor or inhibiting tumor growth in a patient, comprising administering to the patient an effective amount of an siRNA compound, wherein the siRNA compound comprises at least a portion that hybridizes to an CPT1C: transcript under physiological conditions and decreases the expression of CPT1C in a tumor cell.

28-35. (canceled)

36. A method for treatment of tumor cells in a mammalian subject, the method comprising the step of administering to the subject a therapeutic agent effective to reduce the effective amount of CPT1C in the tumor cells.

37-39. (canceled)

40. A method for treating tumor cells in an individual suffering from a cancer that expresses CPT1C in amounts higher than in normal tissue of the same type or depends on CPT1C for survival under hypoxic conditions, comprising administering to the individual a composition effective to inhibit expression of CPT1C by the tumor cells and increase apoptosis or reduce proliferation in the tumor cells.

41-47. (canceled)

48. A method for treating a cancer patient, the method comprising:
(a) identifying cancer cells in the patient that have upregulated expression of CPT1C or contain a level of a substance associated with higher than normal CPT1C activity; and
(b) administering to the individual a composition effective to inhibit expression of CPT1C by the cancer cells.

49-55. (canceled)

56. A method for treating cancer in a subject which comprises administering a nucleic acid comprising a promoter operatively linked to a nucleic acid sequence of interest wherein the promoter is known to be up-regulated in cancer cells, and wherein the nucleic acid sequence of interest down-regulates expression of CPT1C resulting in growth suppression or death of the cancerous cells.

57-62. (canceled)

63. A method of screening for compounds that down-regulate expression of CPT1C, said method comprising: a) contacting a nucleic acid molecule comprising a promoter from a CPT1C gene operatively linked to a reporter gene with a candidate compound; and b) assessing the level of expression of the reporter gene.

64-68. (canceled)

69. A method for identifying a potential anti-cancer agent which comprises: (a) contacting a cell with the agent wherein the cell comprises a nucleic acid comprising a CPT1C promoter operatively linked to a reporter gene; (b) measuring the level of reporter gene expression in the cell; and (c) comparing the expression level measured in step (h) with the reporter gene expression level measured in an identical cell in the absence of the agent, wherein a lower expression level measured in the presence of the agent is indicative of a potential anti-cancer agent.

70-74. (canceled)

75. A method for identifying a potential anticancer agent comprising: (i) operatively linking a CPT1C promoter with a reporter gene of interest; (ii) introducing the resulting expression cassette into a target cell; (iii) contacting the target cell with a candidate agent; and (iv) comparing the level of reporter gene expression in the presence and absence of the agent, wherein a potential anticancer agent is one that produces a measurable decrease in the level of reporter gene expression in the presence of the agent.

76. (canceled)

77. A method for identifying a potential anticancer agent comprising: (i) operatively linking a p53-responsive element of an intronic sequence of a CPT1C gene with a reporter gene of interest; (ii) introducing the resulting expression cassette into a target cell; (iii) contacting the target cell with a candidate agent; and (iv) comparing the level of reporter gene expression in the presence and absence of the agent, wherein a potential anticancer agent is one that produces a measurable decrease in the level of reporter gene expression in the presence of the agent.

78. (canceled)

79. The method of claim 77, wherein for step (iii), the target cell is contacted with a candidate agent under conditions in which p53 is produced or is present in the cell.

80. (canceled)

81. A method for screening and identifying a compound that is capable of decreasing cellular levels of CPT1C, comprising: a) exposing cells to the compound to be screened; and b) determining whether the compound acts upon the DNA motifs that regulate the CPT1C gene, wherein the +1 position is the transcription start of the gene, to decrease gene expression, thereby identifying a compound capable of decreasing cellular levels CPT1C.

82-84. (canceled)

85. A method of screening for a candidate substance as an anticancer agent that regulates activity of the CPT1C promoter, the method comprising a step selected from the group consisting of: (a) contacting a nucleic acid comprising a CPT1C promoter with a CPT1C promoter binding protein and the candidate substance under conditions that allow binding between the protein and the promoter and determining whether the candidate compound modulates the binding between the protein and the promoter; and (b) contacting the candidate substance with a cell comprising the CPT1C promoter operably attached to a reporter gene coding for an expression product and assaying for expression of the reporter gene expression product.

86. (canceled)

87. A method of screening anti-cancer agents for treating a human, comprising: (a) contacting a mammalian CPT1C protein with a test agent thought to be effective in inhibiting the activity of said protein in the presence of a fatty acyl-Co A known to be a substrate of said protein; (b) determining if said test agent inhibits the activity of said protein, wherein determining if said test agent inhibits the activity of said protein comprises quantitating the amount of fatty acyl-carnitine produced in the presence of said agent; and (c) classifying said test agent as a potential anti-cancer agent if said test agent inhibits the activity of said protein.

88-94. (canceled)
